(19) United States
(12) Patent Application Publication
(10) Pub. No.: US 2021/0046041 A1
(43) Pub. Date: Feb. 18, 2021

(54) DEVICE AND METHOD FOR ADMINISTERING AN ACTIVE INGREDIENT

(71) Applicant: CANOPY GROWTH CORPORATION, Smiths Falls (CA)

(72) Inventor: Kurt LEVY, Dillon, CO (US)

(21) Appl. No.: 16/978,091

(22) PCT Filed: Mar. 5, 2019
(86) PCT No.: PCT/IB2019/000214

§ 371 Date: Sep. 3, 2020

Related U.S. Application Data
(60) Provisional application No. 62/639,225, filed on Mar. 6, 2018.
(60) Provisional application No. 62/639,255, filed on Mar. 6, 2018.

(51) Int. Cl. A61K 31/352; A61M 15/00; A61K 31/05; A61K 31/192; A61K 31/343; A61K 31/355; A61K 31/215; A61K 31/045; A61K 31/015; A61K 31/125; A61K 31/35; A61K 31/01; A61K 31/085

(52) U.S. Cl. CPC A61K 31/352; A61M 15/0003; A61K 31/05; A61K 31/192; A61K 31/343; A61K 31/355; A61K 31/085; A61K 31/045; A61K 31/015; A61K 31/125; A61K 31/35; A61K 31/01; A61K 31/215

(57) ABSTRACT

This disclosure pertains to devices and methods for administering one or more active ingredients, e.g., cannabinoids, terpenes, etc. In particular, the devices and methods disclosed herein administer one or more cannabinoids in a mist for transmucosal and transdermal absorption. In one embodiment, the device contains compositions comprising purified active ingredients (e.g., cannabinoids, terpenes, etc.) and/or unique combinations thereof for delivery via a mist for transmucosal and transdermal adsorption. In one embodiment, the device contains two separate compositions in separate chambers, wherein each composition comprises one or more of the active ingredients. In one embodiment, a composition in one chamber comprises a purified cannabinoid and the composition in the other chamber comprises a purified terpene. In an embodiment, the device operates without heating the active ingredients.

### CROSS-REFERENCE TO RELATED APPLICATIONS

This application claims the benefit of and priority to U.S. Provisional Patent Application No. 62/639,225 filed on Mar. 6, 2018 and U.S. Provisional Patent Application No. 62/639,255 filed on Mar. 6, 2018, each of which is hereby incorporated by reference in its entirety.

### TECHNICAL FIELD

This disclosure relates to the formulation and drug industry. In particular, this disclosure pertains to devices and methods for administering one or more active ingredients.

### BACKGROUND

Many compositions, methods, apparatuses, and devices have been developed for administering active ingredients, for example cannabinoids. The use of devices and related methods for administering active ingredients is old and developing.

One of the most common and well-known devices and methods for administering active ingredients involves using a water pipe. After filling a water pipe with water, dried plant material, e.g., cannabis, tobacco, etc., is ignited and the smoke is filtered through the water and a user inhales the filtered smoke. The use of a water pipe poses several problems.

First, the inhalation of smoke can harm the lungs and can lead to several other health issues. Also, heat can cause unwanted chemical changes and reactions creating unwanted compounds and by-products. Second, most dried plant material is not administrable in precise, controlled doses. For example, cannabis plants are bred to have certain ratios and concentrations of cannabinoids but rarely in the exact amounts needed or wanted. Third, a water pipe is often bulky and cumbersome to store and use, thereby making a water pipe an inconvenient device for everyday use.

A more recently developed device and method for administering active ingredients involves using an electronic cigarette. Instead of igniting a natural product, e.g., tobacco or cannabis, an electronic device atomizes a liquid formulation comprising one or more active ingredients, creating a vapor that a user inhales. However, a number of issues still exist with electronic cigarettes. Electronic cigarettes are relatively new and the health risks are unknown. While electronic cigarettes do not contain the number of toxic chemicals as found in natural cigarettes, electronic cigarettes still pose potential health issues, e.g., contamination of the liquid formulation, device malfunctioning, potential secondary effects to passerbys, exploding, etc. An electronic cigarette also heats the active ingredients which can cause potential reactions, e.g., changing the chemical structure and overall efficacy of the active ingredients.

There exists a need for devices and methods for administering one or more active ingredients without heating the active ingredients. There also exists a need for devices and methods for administering one or more active ingredients not involving plant matter. In particular, there exists a need for devices and methods for administering cannabinoids.

Cannabinoids are of particular interest for administration for a number of desired effects. For example, tetrahydrocannabinol (THC) is useful for relieving pain, treating glaucoma, and relieving nausea. Conventionally, most administrations of cannabinoids involve utilizing plant matter as the source of cannabinoids. This poses several problems. One of the main issues is that using plant matter does not allow a user to control the dosage of individual cannabinoids. While cannabis plants can be bred to produce more cannabinoids over others, there is almost no guarantee that the plant has the desired cannabinoid profile without some type of analytical test.

There exists a need for devices and methods for administering one or more active ingredients in specific doses. There exists an unmet need for a fast and efficient device and method for administering one or more active ingredients, e.g., cannabinoids and terpenes. There exists a need to administer one or more active ingredients without producing and/or inhaling smoke.

### SUMMARY

The present disclosure provides new devices, and methods and uses employing these devices, for administering one or more active ingredients to a subject. In an embodiment, the active ingredients include one or more cannabinoids and/or terpenes.

In an embodiment, the present disclosure relates to a device for administering an aqueous composition, the device comprising: (i) a container comprising a first chamber and a second chamber; wherein the first chamber contains a first composition comprising an aqueous acid; wherein the second chamber contains a second composition comprising an aqueous carbonate base; wherein at least one of the first composition and the second composition comprises an active ingredient; (ii) a means for expelling the first composition from the first chamber; (iii) a means for expelling the second composition from the second chamber; and (iv) a nozzle.

In an embodiment, the present disclosure relates to a method of administering an aqueous composition, comprising: providing a device comprising: (i) a container comprising a first chamber and a second chamber; wherein the first chamber contains a first composition comprising an aqueous acid; wherein the second chamber contains a second composition having an aqueous carbonate base; wherein at least one of the first composition and the second composition comprises an active ingredient; (ii) a means for expelling the first composition from the first chamber; (iii) a means for expelling the second composition from the second chamber; and (iv) a nozzle, expelling the first composition from the first chamber; expelling the second composition from the second chamber; and administering the first composition and the second composition through the nozzle.

Further embodiments, advantages, permutations and combinations of the present disclosure will be apparent from the above and from the following detailed description of the various particular embodiments, all of which is intended to be non-limiting.

### DESCRIPTION

The present disclosure relates to new devices, and methods and uses employing these devices, for administering one or more active ingredients.

In one aspect, disclosed herein are new devices for administering one or more active ingredients. The active ingredients may be one or more of any number of active ingredients as described further herein. In an embodiment, the active ingredients are one or more cannabinoids.

In one embodiment, the devices disclosed herein do not heat the one or more active ingredients. By this, it is meant that no heat is applied to the active ingredients or the composition comprising the active ingredients within the device. In an embodiment, the device disclosed herein does not comprise any form of a heating component or element (e.g., an atomizer). In an embodiment, external heat is not used in the operation of the device disclosed herein. In an embodiment, heat may be applied to a composition within the device that does not comprise an active ingredient (e.g. a chamber of the device that does not contain the active ingredient). In such embodiments, if heat is applied to the chamber that does not contain the active ingredient, the heat does not transfer to the chamber comprising the active ingredient.

In one embodiment, the devices disclosed herein administer the one or more active ingredients from a container. In one embodiment, the devices disclosed herein contain the one or more active ingredients in separate containers or separate chambers within a container.

In one embodiment, the devices disclosed herein administer one active ingredient. The active ingredient may be comprised in a single composition within the device or may be comprised within two or more separate compositions within the device.

In one embodiment, the devices disclosed herein administer two or more active ingredients at the same time. In an embodiment, the two or more active ingredients are within the same container or the same chamber within a container. In an embodiment, the two or more active ingredients are in separate containers or separate chambers within a container. In one embodiment, the devices disclosed herein administer two or more compositions at the same time. In one embodiment, the devices disclosed herein administer two or more compositions one after the other.

In an embodiment, the device disclosed herein administers one or more active ingredients in a precise dose. By “precise dose”, it is meant that the device administers a desired and controlled amount of the active ingredient(s). In an embodiment, the precise dose is administered based on a controlled concentration of active ingredient within a composition contained within the device. In an embodiment, the active ingredients are purified and are at a specific concentration within a composition in the device. In an embodiment, an expelling means of the device regulates the dose of the active ingredient. This may be performed, for example, through a controlled volume delivery of the active ingredient from the device to the subject. In an embodiment, the subject may adjust the precise dose to a desired quantity (e.g., by adjusting the delivery volume).

In some embodiments, the devices disclosed herein are for administering cannabinoids. In one embodiment, the devices disclosed herein administer cannabinoids in precise doses. In one embodiment, the devices disclosed herein administer cannabinoids with other active ingredients, e.g., terpenes. In one embodiment, the devices disclosed herein administer cannabinoids without producing smoke. In one embodiment, the devices disclosed herein administer cannabinoids without heating the cannabinoids.

In an embodiment, the device disclosed herein is for the administering of one or more active ingredients in an aqueous composition. By “aqueous composition”, it is meant that the composition is one in which the solvent is or comprises water (i.e. the continuous phase that dissolves or suspends the other components is water). As one will appreciate, acids and bases are aqueous mediums.

In one embodiment, disclosed herein is a new device for administering an aqueous composition, comprising:
a Container comprising a First Chamber and a Second Chamber;
wherein the First Chamber contains a First Composition comprising an Aqueous Acid;
wherein the Second Chamber contains a Second Composition comprising an Aqueous Carbonate Base;
wherein at least one of the First Composition and the Second Composition comprises an Active Ingredient;
a means for expelling the First Composition from the First Chamber;
a means for expelling the Second Composition from the Second Chamber; and
a Nozzle.

As used herein, the term “Container” refers to a receptacle, vessel, and/or repository capable of containing a substance. In one embodiment, a Container comprises an opening. In one embodiment, a Container comprises a means for closing an opening, e.g., a cap, a lid, a cover, etc. In one embodiment, a Container does not have an opening. In one embodiment, the Container is capable of being ruptured or punctured to access the active ingredient or composition contained therein for administration. In one embodiment, a Container is composed of plastic, e.g., polyethylene, polypropylene, rubber, etc. In one embodiment, a Container is composed of glass. In one embodiment, a Container is composed of compostable material.

As used herein, the term “Chamber” refers to an opening, a space, and/or a cavity within a structure, such as the Container as described herein. In one embodiment, a Container comprises a First Chamber. In one embodiment, a Container comprises a First Chamber and a Second Chamber. In one embodiment, a Chamber contains a composition. In one embodiment, a First Chamber contains a First Composition and a Second Chamber contains a Second Composition. In one embodiment, a First Chamber and a Second Chamber are connected by an opening. In one embodiment, a First Chamber and a Second Chamber are separated from one another within a Container. In one embodiment, the Chamber is capable of being ruptured or punctured to access the active ingredient or composition contained therein for administration. In one embodiment, a Chamber is composed of plastic, e.g., polyethylene, polypropylene, rubber, etc. In one embodiment, a Chamber is composed of glass. In one embodiment, a Chamber is composed of compostable material. In one embodiment, a Chamber contains a bag.

As used herein, the term “bag” refers to a flexible receptacle. In one embodiment, a bag is composed of plastic, e.g., polyethylene, polypropylene, rubber, etc. In one embodiment, a bag is composed of leather. In one embodiment, a bag is composed of cloth. In one embodiment, a bag contains a Composition. In one embodiment, a bag contains an opening. In one embodiment, a bag contains a means for closing an opening, e.g., a cover, a plug, a cap, etc. In one embodiment, the bag is capable of being ruptured or punctured to access the active ingredient or composition contained therein for administration.

As used herein, the term “Composition” refers to a formulation comprising one or more compounds. In one embodiment, a Composition comprises an aqueous solution. In one embodiment, a Composition comprises an Active Ingredient. In one embodiment, a Composition comprises two or more Active Ingredients. In one embodiment, a Composition comprises a concentration of an Active Ingredient or Active Ingredients, e.g., measured by moles per volume. In one embodiment, a Composition comprises compounds preventing the degradation of other compounds such as Active Ingredients, e.g., stabilizers, pH buffers, etc. In one embodiment, a Composition comprises a bulking agent, i.e., a non-Active Ingredient.

As used herein, the term “Aqueous Acid” refers to a solution comprising water acting as a solvent and an acid. Within the context of this disclosure, the term “acid” refers to a chemical species donating protons or hydrogen ions and/or accepting electrons. In one embodiment, an acid is a compound lowering the pH of a solution below 7. In some embodiments, a compound may have both acidic and basic qualities. In one embodiment, an Aqueous Acid comprises glucono delta-lactone, citric acid, ascorbic acid, acetic acid, lactic acid, malic acid, tartaric acid, potassium citrate, and/or sodium citrate.

As used herein, the term “glucono delta-lactone” refers to a compound with the following structural formula:

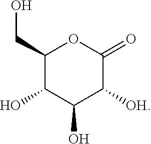 [SMILES: O=C1O[C@H](CO)[C@@H](O)[C@H](O)[C@H]1O]

Glucono delta-lactone is also referred to as “gluconolatone”. Glucono delta-lactone is often characterized as a sequestrant, an acidifier, or a curing, pickling, or leavening agent.

As used herein, the term “citric acid” refers to a compound with the following structural formula:

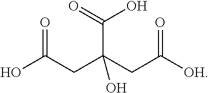 [SMILES: O=C(O)CC(O)(CC(=O)O)C(=O)O]

Citric acid naturally occurs in citrus fruits. Citric acid is often characterized as an acidifier and a flavoring and chelating agent.

As used herein, the term “ascorbic acid” refers to a compound of the following structural formula:

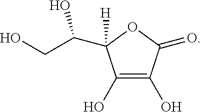 [SMILES: O=C1O[C@H]([C@@H](O)CO)C(O)=C1O]

Ascorbic acid is also commonly known as Vitamin C.

As used herein, the term “acetic acid” refers to a compound with the following structural formula:

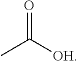

Acetic acid is often used as a chemical reagent for producing other chemical compounds. Acetic acid is used in the production of vinyl acetate monomer.

As used herein, the term “lactic acid” refers to a compound with the following structural formula:

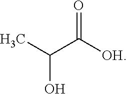

In one embodiment, lactic acid is a white solid that is water-soluble. In one embodiment, lactic acid is a clear liquid. Lactic acid is produced both naturally and synthetically. Within the context of this disclosure, lactic acid comprises one of its chiral forms, a mixture, or a racemic mixture of all its forms.

As used herein, the term “malic acid” refers to a compound of the following structural formula:

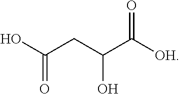

Within the context of this disclosure, the term “malic acid” may refer to either a single isomeric form or a racemic mixture.

As used herein, the term “tartaric acid” refers to a compound with the following structural formula:

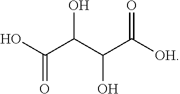

Within the context of this disclosure, the term “tartaric acid” may refer to either a single isomeric form or a racemic mixture.

As used herein, the term “potassium citrate” refers to a compound with the following structural formula:

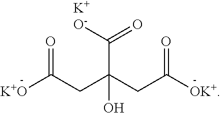

In one embodiment, potassium citrate is a white, crystalline powder. In one embodiment, potassium citrate is a food additive, e.g., regulating acidity.

As used herein, the term “sodium citrate” refers to a class of compounds of sodium salts of citrate.

In one embodiment, sodium citrate refers to monosodium citrate and has the following structural formula:

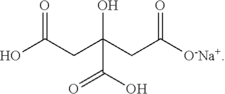

In one embodiment, sodium citrate refers to disodium citrate and has the following structural formula:

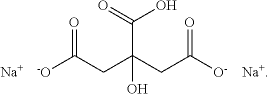

In one embodiment, sodium citrate refers to trisodium citrate and has the following structural formula:

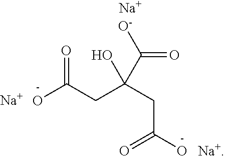 [SMILES: [Na+] ; [Na+] ; O=C(O)CC(O)(CC(=O)O)C(=O)[O-]]

As used herein, the term “Aqueous Carbonate Base” refers to a solution in which water is a solvent and comprises a carbonate or a bicarbonate. In one embodiment, an Aqueous Carbonate Base is a solution with a pH above 7.

As used herein, the term “carbonate” refers to the dianionic polyatomic compound with the following structural formula:

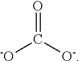

In one embodiment, the carbonate is decarboxylated releasing CO₂ gas.

As used herein, the term “bicarbonate” refers to a compound with the following structural formula:

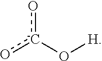

In one embodiment, the bicarbonate is decarboxylated releasing CO₂ gas.

In one embodiment, an Aqueous Acid and an Aqueous Carbonate Base react with one another to make an effervescent.

As used herein, the term “effervescent” refers to a compound causing the escape of gas from an aqueous compound. One visual sign of effervescence is bubbles or fizzing. In one embodiment, the effervescent is a base reacting with an acid. In one embodiment, the effervescent is an acid reacting with a base. In one embodiment, administering one or more Active Ingredients via an effervescent creates an efficient delivery mechanism for transmucosal and transdermal because the effervescent acts as an effective penetration enhancer. In an embodiment, the effervescent causes an aqueous composition to form a mist. In an embodiment, the mist will contain an Active Ingredient. In an embodiment, the Active Ingredient is one or more cannabinoids and the mist will comprise the one or more cannabinoids.

As used herein, the term “transmucosal” has its plain and ordinary meaning in that it refers to a route of administration in which the drug is diffused through the mucous membrane. This can refer to oral (e.g. buccal, sublingual, etc), nasal, vaginal, rectal, or ocular routes. In an embodiment, transmucosal refers to delivery by inhalation into the oral cavity and contact with mucosal surfaces therein, such as buccal and/or sublingual.

As used herein, the term “transdermal” has its plain and ordinary meaning in that it refers to a route of administration wherein active ingredients are delivered across the skin, e.g. for local or systemic distribution. In an embodiment, transdermal includes a transdermal patch.

As used herein, the term “a means for expelling” refers to a method, device, apparatus, etc., for distributing a solution. In one embodiment, a means for expelling comprises a pump system for drawing a solution from a source (a reservoir, cavity, Chamber, etc.) through a pipe and through a Nozzle. In one embodiment, a means for expelling comprises one end of a Tube connected to a Nozzle and the other end within a Chamber containing a Composition. In one embodiment, a means for expelling comprises aerosolizing a Composition into a mist suitable for transmucosal absorption.

As used herein, the term “Nozzle” refers to a device controlling the distribution and flow of a fluid, solution, or composition. In one embodiment, a Nozzle connects to a tube. In one embodiment, a Nozzle connects to a pipe. In one embodiment, a Nozzle connects to a Chamber. In one embodiment, a Nozzle is a tube. In one embodiment, a Nozzle is an atomizer nozzle. In one embodiment, a Nozzle controls the distribution of a solution, e.g., a fine mist with a large distribution, a strong stream within a small radius, a mist with a small travel distance, etc.

As used herein, the term “Active Ingredient” refers to a biologically active compound causing an effect or effects. In one embodiment, an Active Ingredient causes a chemical reaction within an organism. In one embodiment, an Active Ingredient binds to a neurotransmitter receptor within an organism. In one embodiment, the Compositions disclosed herein comprise an Active Ingredient. In one embodiment, the Compositions disclosed herein comprise two or more Active Ingredients. In one embodiment, an Active Ingredient comprises about 0-10% of the mass % of a Composition. In one embodiment, an Active Ingredient comprises about 0-5% of the mass % of a Composition. In one embodiment, an Active Ingredient comprises about 0-2.5% of the mass % of a Composition. In one embodiment, an Active Ingredient is a plant extract. In one embodiment, an Active Ingredient is an isolated or purified compound. In one embodiment, an Active Ingredient is a formulation.

In one embodiment, an Active Ingredient is a cannabinoid. In an embodiment, each of the First Composition and the Second Composition may individually comprise one or more cannabinoids. In an embodiment, only one of the First Composition or the Second Composition comprises cannabinoid(s). In an embodiment, only one of the First Composition or the Second Composition comprises cannabinoid(s), and it is only a single type of cannabinoid (e.g., a first cannabinoid). In an embodiment, each of the First Composition and the Second Composition comprise only a single cannabinoid, and that cannabinoid may be the same in both (e.g. a first cannabinoid) or different (e.g., a first cannabinoid in one and a second cannabinoid in the other).

As used herein, the term “cannabinoid” refers to a compound belonging to a class of secondary compounds commonly found in plants of genus cannabis. Plants of genus cannabis include several species, including Cannabis sativa, Cannabis indica, and Cannabis ruderalis. There is a long history of cultivating plants of genus cannabis for hemp fibers, seeds and seed oils, medicinal purposes, and recreational activities.

In one embodiment, a cannabinoid is found in a plant, e.g., a plant of genus cannabis, and is sometimes referred to as a phytocannabinoid. In one embodiment, a cannabinoid is found in a mammal, sometimes called an endocannabinoid. In one embodiment, a cannabinoid is made in a laboratory setting, sometimes called a synthetic cannabinoid or a semi-synthetic cannabinoid. For example, the manufacture of cannabinoid compounds and their analogs or derivatives using semi-synthetic means involves contacting an appropriate cannabinoid substrate with one of the cannabinoid synthase enzymes (e.g. tetrahydrocannabinolic acid (THCA) from canabigerolic acid (CBGA) with THC synthase). Other means for synthetic or semi-synthetic manufacture of cannabinoids may involve cell culture of cells that produce cannabinoids.

In one embodiment, a cannabinoid acts upon a cellular receptor, such as a G-coupled protein receptor (e.g., a serotonin receptor, a cannabinoid receptor, TRPV1, an opioid receptor, etc.) thereby causing a response on the brain or body. In one embodiment, a cannabinoid affects the activity of other compounds at one or more receptors by acting as an agonist, partial agonist, inverse agonist, antagonist, etc.

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C₄ (CBD-C₄), Cannabidivarinic Acid (CBDVA), Cannabidivarin (CBDV), Cannabidiorcol (CBD-C₁), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C₄ (THCA-C₄), Tetrahydrocannbinol C₄ (THC-C₄), Tetrahydrocannabivarinic acid (THCVA), Tetrahydrocannabivarin (THCV), Tetrahydrocannabiorcolic acid (THCA-C₁), Tetrahydrocannabiorcol (THC-C₁), Δ7-cis-iso-tetrahydrocannabivarin, Δ8-tetrahydrocannabinolic acid (Δ8-THCA), Cannabivarinodiolic (CBNDVA), Cannabivarinodiol (CBNDV), Δ8-tetrahydrocannabinol (Δ8-THC), Δ9-tetrahydrocannabinol (Δ9-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA), Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C₄ (CBN-C₄), Cannabivarin (CBV), Cannabino-C₂ (CBN-C₂), Cannabiorcol (CBN-C₁), Cannabinodiol (CBND), Cannabinodiolic Acid (CBNDA), Cannabinodivarin (CBDV), Cannabitriol (CBT), 10-Ethoxy-9-hydroxy-Δ6a-tetrahydrocannabinol, 8,9-Dihydroxy-Δ6a(10a)-tetrahydrocannabinol (8,9-Di-OH-CBT-C₅), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, and Dodeca-2E,4E-dienoic acid isobutylamide.

In one embodiment, the one or more cannabinoids are chosen from THC, Δ9-THC, Δ8-THC, THCA, THCV, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

As used herein, the term “THC” refers to tetrahydrocannabinol and has the following structural formula:

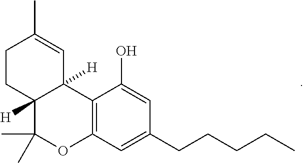 [SMILES: CCCCCc1cc(O)c2c(c1)OC(C)(C)[C@@H]1CCC(C)=C[C@@H]21]

In an embodiment, within the context of this disclosure, THC may be present in one or more of an aqueous Composition, for administration via an effervescent delivery mechanism to a transmucosal or transdermal surface, e.g. as a mist.

As used herein, the term “THCA” refers to tetrahydrocannabinolic acid and has the following structural formula:

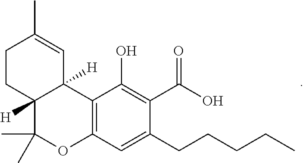 [SMILES: CCCCCc1cc2c(c(O)c1C(=O)O)[C@@H]1C=C(C)CC[C@H]1C(C)(C)O2]

Decarboxylating THCA with heat, light, etc., forms THC, Δ8-THC, and other potential cannabinoids. In an embodiment, within the context of this disclosure, THCA may be present in one or more of an aqueous Composition, for administration via an effervescent delivery mechanism to a transmucosal or transdermal surface, e.g. as a mist.

As used herein, the term “THCV” refers to tetrahydrocannabivarin and has the following structural formula:

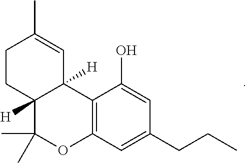 [SMILES: CCCc1cc(O)c2c(c1)OC(C)(C)[C@@H]1CCC(C)=C[C@@H]21]

In an embodiment, within the context of this disclosure, THCV may be present in one or more of an aqueous Composition, for administration via an effervescent delivery mechanism to a transmucosal or transdermal surface, e.g. as a mist.

As used herein, the term “THCVA” refers to tetrahydrocannabivarinic acid and has the following structural formula:

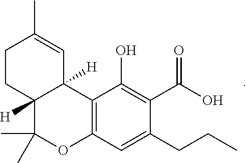 [SMILES: CCCc1cc2c(c(O)c1C(=O)O)[C@@H]1C=C(C)CC[C@H]1C(C)(C)O2]

Decarboxylating THCVA with heat, light, etc., forms THCV, Δ8-THCV, Δ9-THCV, and other possible cannabinoid derivatives. In an embodiment, within the context of this disclosure, THCVA may be present in one or more of an aqueous Composition, for administration via an effervescent delivery mechanism to a transmucosal or transdermal surface, e.g. as a mist.

As used herein, the term “Δ8-THC” refers to delta-8-tetrahydrocannabinol and has the following structural formula:

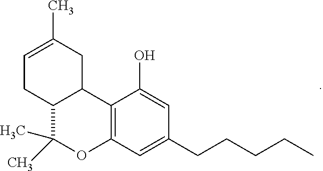

In an embodiment, within the context of this disclosure, Δ8-THC may be present in one or more of an aqueous Composition, for administration via an effervescent delivery mechanism to a transmucosal or transdermal surface, e.g. as a mist.

As used herein, the term “Δ8-THCV” refers to delta-8-tetrahydrocannabivarin and has the following structural formula:

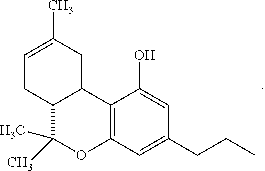

In an embodiment, within the context of this disclosure, Δ8-THCV may be present in one or more of an aqueous Composition, for administration via an effervescent delivery mechanism to a transmucosal or transdermal surface, e.g. as a mist.

As used herein, the term “Δ9-THCV” refers to delta-9-tetrahydrocannabivarin and has the following structural formula:

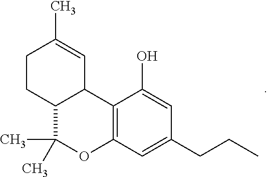

In an embodiment, within the context of this disclosure, Δ9-THCV may be present in one or more of an aqueous Composition, for administration via an effervescent delivery mechanism to a transmucosal or transdermal surface, e.g. as a mist.

As used herein, the term “CBD” refers to cannabidiol and has the following structural formula:

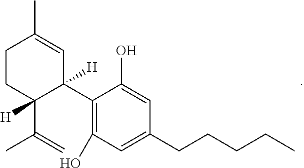 [SMILES: C=C(C)[C@@H]1CCC(C)=C[C@H]1c1c(O)cc(CCCCC)cc1O]

In an embodiment, within the context of this disclosure, CBD may be present in one or more of an aqueous Composition, for administration via an effervescent delivery mechanism to a transmucosal or transdermal surface, e.g. as a mist.

As used herein, the term “CBDA” refers to cannabidiolic acid and has the following structural formula:

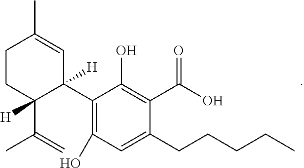 [SMILES: C=C(C)[C@@H]1CCC(C)=C[C@H]1c1c(O)cc(CCCCC)c(C(=O)O)c1O]

Decarboxylating CBDA with heat, light, etc., forms CBD and other possible cannabinoid derivatives. In an embodiment, within the context of this disclosure, CBDA may be present in one or more of an aqueous Composition, for administration via an effervescent delivery mechanism to a transmucosal or transdermal surface, e.g. as a mist.

As used herein, the term “CBDV” refers to cannabidivarin and has the following structural formula:

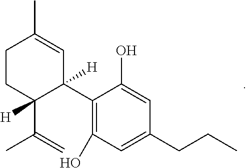 [SMILES: C=C(C)[C@@H]1CCC(C)=C[C@H]1c1c(O)cc(CCC)cc1O]

In an embodiment, within the context of this disclosure, CBDV may be present in one or more of an aqueous Composition, for administration via an effervescent delivery mechanism to a transmucosal or transdermal surface, e.g. as a mist.

As used herein, the term “CBDVA” refers to cannabidivarinic acid and has the following structural formula:

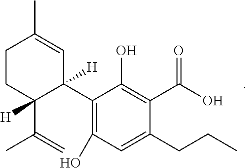 [SMILES: C=C(C)[C@@H]1CCC(C)=C[C@H]1c1c(O)cc(CCC)c(C(=O)O)c1O]

Decarboxylating CBDVA with heat, light, etc., forms CBDV and other possible cannabinoid derivatives. In an embodiment, within the context of this disclosure, CBDVA may be present in one or more of an aqueous Composition, for administration via an effervescent delivery mechanism to a transmucosal or transdermal surface, e.g. as a mist.

As used herein, the term “CBC” refers to cannabichromene and has the following structural formula:

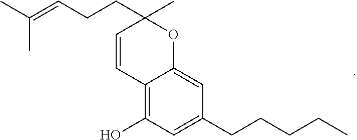 [SMILES: CCCCCc1cc(O)c2c(c1)OC(C)(CCC=C(C)C)C=C2]

In an embodiment, within the context of this disclosure, CBC may be present in one or more of an aqueous Composition, for administration via an effervescent delivery mechanism to a transmucosal or transdermal surface, e.g. as a mist.

As used herein, the term “CBCA” refers to cannabichromenic acid and has the following structural formula:

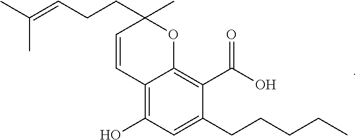 [SMILES: CCCCCc1cc(O)c2c(c1C(=O)O)OC(C)(CCC=C(C)C)C=C2]

Decarboxylating CBCA with heat, light, etc., forms CBC and other possible cannabinoid derivatives. In an embodiment, within the context of this disclosure, CBCA may be present in one or more of an aqueous Composition, for administration via an effervescent delivery mechanism to a transmucosal or transdermal surface, e.g. as a mist.

As used herein, the term “CBCV” refers to cannabichromevarin and has the following structural formula:

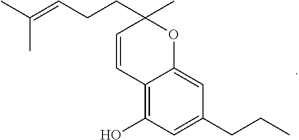 [SMILES: CCCc1cc(O)c2c(c1)OC(C)(CCC=C(C)C)C=C2]

In an embodiment, within the context of this disclosure, CBCV may be present in one or more of an aqueous Composition, for administration via an effervescent delivery mechanism to a transmucosal or transdermal surface, e.g. as a mist.

As used herein, the term “CBCVA” refers to cannabichromevarinic acid and has the following structural formula:

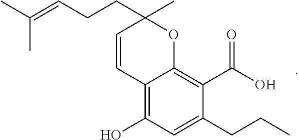 [SMILES: CCCc1cc(O)c2c(c1C(=O)O)OC(C)(CCC=C(C)C)C=C2]

Decarboxylating CBCVA with heat, light, etc., forms CBCV and other possible cannabinoid derivatives. In an embodiment, within the context of this disclosure, CBCVA may be present in one or more of an aqueous Composition, for administration via an effervescent delivery mechanism to a transmucosal or transdermal surface, e.g. as a mist.

As used herein, the term “CBG” refers to cannabigerol and has the following structural formula:

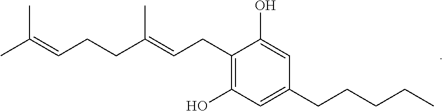 [SMILES: CCCCCc1cc(O)c(C/C=C(\C)CCC=C(C)C)c(O)c1]

In an embodiment, within the context of this disclosure, CBG may be present in one or more of an aqueous Composition, for administration via an effervescent delivery mechanism to a transmucosal or transdermal surface, e.g. as a mist.

As used herein, the term “CBGA” refers to cannabigerolic acid and has the following structural formula:

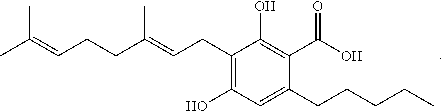 [SMILES: CCCCCc1cc(O)c(C/C=C(\C)CCC=C(C)C)c(O)c1C(=O)O]

Decarboxylating CBGA with heat, light, etc., forms CBG and other possible cannabinoid derivatives. In an embodiment, within the context of this disclosure, CBGA may be present in one or more of an aqueous Composition, for administration via an effervescent delivery mechanism to a transmucosal or transdermal surface, e.g. as a mist.

As used herein, the term “CBGV” refers to cannabigerovarin and has the following structural formula:

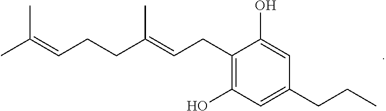 [SMILES: CCCc1cc(O)c(C/C=C(\C)CCC=C(C)C)c(O)c1]

In an embodiment, within the context of this disclosure, CBGV may be present in one or more of an aqueous Composition, for administration via an effervescent delivery mechanism to a transmucosal or transdermal surface, e.g. as a mist.

As used herein, the term “CBGVA” refers to cannabigerovarinic acid and has the following structural formula:

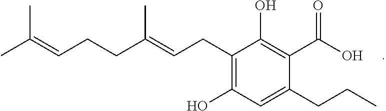 [SMILES: CCCc1cc(O)c(C/C=C(\C)CCC=C(C)C)c(O)c1C(=O)O]

Decarboxylating CBGVA with heat, light, etc., forms CBGV and other possible cannabinoid derivatives. In an embodiment, within the context of this disclosure, CBGVA may be present in one or more of an aqueous Composition, for administration via an effervescent delivery mechanism to a transmucosal or transdermal surface, e.g. as a mist.

As used herein, the term “CBN” refers to cannabinol and has the following structural formula:

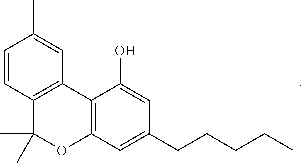 [SMILES: CCCCCc1cc(O)c2c(c1)OC(C)(C)c1ccc(C)cc1-2]

In an embodiment, within the context of this disclosure, CBN may be present in one or more of an aqueous Composition, for administration via an effervescent delivery mechanism to a transmucosal or transdermal surface, e.g. as a mist.

As used herein, the term “CBNA” refers to cannabinolic acid and has the following structural formula:

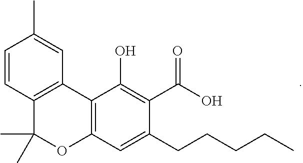 [SMILES: CCCCCc1cc2c(c(O)c1C(=O)O)-c1cc(C)ccc1C(C)(C)O2]

Decarboxylating CBNA with heat, light, etc., forms CBN and other possible cannabinoid derivatives. In an embodiment, within the context of this disclosure, CBNA may be present in one or more of an aqueous Composition, for administration via an effervescent delivery mechanism to a transmucosal or transdermal surface, e.g. as a mist.

As used herein, the term “CBNV” or “CBV” refers to cannabivarin and has the following structural formula:

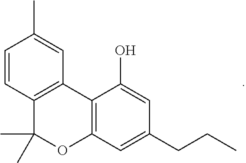 [SMILES: CCCc1cc(O)c2c(c1)OC(C)(C)c1ccc(C)cc1-2]

In an embodiment, within the context of this disclosure, CBNV may be present in one or more of an aqueous Composition, for administration via an effervescent delivery mechanism to a transmucosal or transdermal surface, e.g. as a mist.

As used herein, the term “CBNVA” refers to cannabivarinic acid and has the following structural formula:

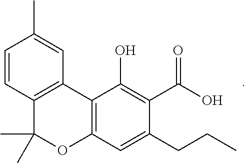 [SMILES: CCCc1cc2c(c(O)c1C(=O)O)-c1cc(C)ccc1C(C)(C)O2]

Decarboxylating CBNVA with heat, light, etc., forms CBNV and other possible cannabinoid derivatives. In an embodiment, within the context of this disclosure, CBNVA may be present in one or more of an aqueous Composition, for administration via an effervescent delivery mechanism to a transmucosal or transdermal surface, e.g. as a mist.

As used herein, the term “CBND” refers to cannabinodiol and has the following structural formula:

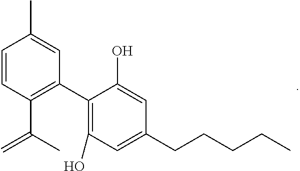 [SMILES: C=C(C)c1ccc(C)cc1-c1c(O)cc(CCCCC)cc1O]

In an embodiment, within the context of this disclosure, CBND may be present in one or more of an aqueous Composition, for administration via an effervescent delivery mechanism to a transmucosal or transdermal surface, e.g. as a mist.

As used herein, the term “CBNDA” refers to cannabinodiolic acid and has the following structural formula:

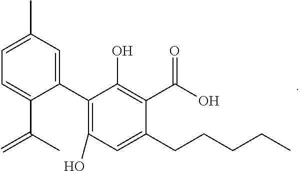 [SMILES: C=C(C)c1ccc(C)cc1-c1c(O)cc(CCCCC)c(C(=O)O)c1O]

Decarboxylating CBNDA with heat, light, etc., forms CBND and other possible cannabinoid derivatives. In an embodiment, within the context of this disclosure, CBNDA may be present in one or more of an aqueous Composition, for administration via an effervescent delivery mechanism to a transmucosal or transdermal surface, e.g. as a mist.

As used herein, the term “CBNDV” refers to cannabivarinodiol and has the following structural formula:

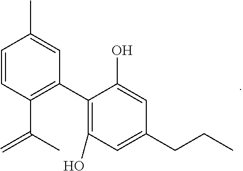 [SMILES: C=C(C)c1ccc(C)cc1-c1c(O)cc(CCC)cc1O]

In an embodiment, within the context of this disclosure, CBNDV may be present in one or more of an aqueous Composition, for administration via an effervescent delivery mechanism to a transmucosal or transdermal surface, e.g. as a mist.

As used herein, the term “CBNDVA” refers to cannabivarinodiolic acid and has the following structural formula:

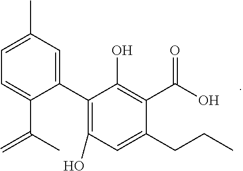 [SMILES: C=C(C)c1ccc(C)cc1-c1c(O)cc(CCC)c(C(=O)O)c1O]

Decarboxylating CBNDVA with heat, light, etc., forms CBNDV and other possible cannabinoid derivative. In an embodiment, within the context of this disclosure, CBNDVA may be present in one or more of an aqueous Composition, for administration via an effervescent delivery mechanism to a transmucosal or transdermal surface, e.g. as a mist.

As used herein, the term “CBL” refers to cannabicyclol and has the following

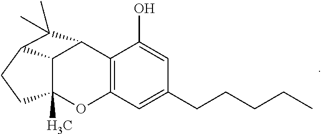

In an embodiment, within the context of this disclosure, CBL may be present in one or more of an aqueous Composition, for administration via an effervescent delivery mechanism to a transmucosal or transdermal surface, e.g. as a mist.

As used herein, the term “CBLA” refers to cannabicyclolic acid and has the following structural formula:

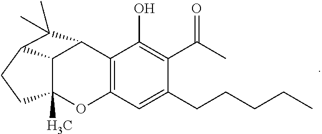

Decarboxylating CBLA with heat, light, etc., forms CBL and other possible cannabinoid derivatives. In an embodiment, within the context of this disclosure, CBLA may be present in one or more of an aqueous Composition, for administration via an effervescent delivery mechanism to a transmucosal or transdermal surface, e.g. as a mist.

As used herein, the term “CBLV” refers to cannabicyclovarin and has the following structural formula:

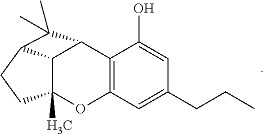

In an embodiment, within the context of this disclosure, CBLV may be present in one or more of an aqueous Composition, for administration via an effervescent delivery mechanism to a transmucosal or transdermal surface, e.g. as a mist.

As used herein, the term “CBLVA” refers to cannabielvarinsoinic acid and has the following structural formula:

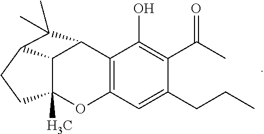

Decarboxylating CBLVA with heat, light, etc., forms CBLV and other possible cannabinoid derivatives. In an embodiment, within the context of this disclosure, CBLVA may be present in one or more of an aqueous Composition, for administration via an effervescent delivery mechanism to a transmucosal or transdermal surface, e.g. as a mist.

As used herein, the term “CBE” refers to cannabielsoin and has the following structural formula:

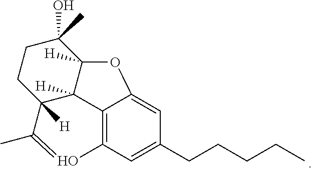

In an embodiment, within the context of this disclosure, CBE may be present in one or more of an aqueous Composition, for administration via an effervescent delivery mechanism to a transmucosal or transdermal surface, e.g. as a mist.

As used herein, the term “CBEA” refers to cannabielsoic acid and has the following structural formula:

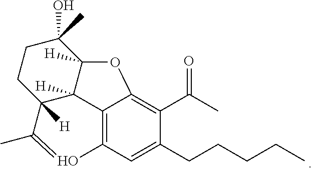

Decarboxylating CBEA with heat, light, etc., forms CBE and other possible cannabinoid derivatives. In an embodiment, within the context of this disclosure, CBEA may be present in one or more of an aqueous Composition, for administration via an effervescent delivery mechanism to a transmucosal or transdermal surface, e.g. as a mist.

As used herein, the term “CBEV” refers to cannabivarinselsoin and has the following structural formula:

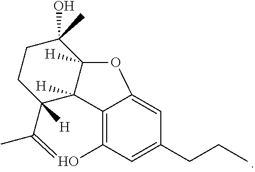

In an embodiment, within the context of this disclosure, CBEV may be present in one or more of an aqueous Composition, for administration via an effervescent delivery mechanism to a transmucosal or transdermal surface, e.g. as a mist.

As used herein, the term “CBEVA” refers to cannabivarinselsoinic acid and has the following structural formula:

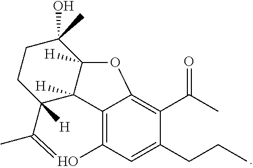

Decarboxylating CBEVA with heat, light, etc., forms CBEV and other possible cannabinoid derivatives. In an embodiment, within the context of this disclosure, CBEVA may be present in one or more of an aqueous Composition, for administration via an effervescent delivery mechanism to a transmucosal or transdermal surface, e.g. as a mist.

In one embodiment, the one or more cannabinoids are purified.

As used within the context of this disclosure, the term “purified” means extracted, isolated, and/or separated from other compounds, formulations, compositions, matter, and/or mass. In one embodiment, the term “purified” refers to a cannabinoid that is separated from the plant matter from which it was derived. In one embodiment, the term “purified” refers to a terpene that is separated from the plant matter from which it was derived.

Within the context of this disclosure, purified compounds may be purposely formulated with other compounds at various levels of purity. For example, depending on the desired outcome, a particular cannabinoid or terpene may be formulated with other molecules when it is 60-65% pure, 65-70% pure, 70-75% pure, 75-80% pure, 80-85% pure, 85-90% pure, 90-95% pure, 95-99% pure, 99-99.9% pure, 99.9+%, or greater than 99% pure. Provided that the ingredients used for purposeful formulation are purified prior to the said purposeful formulation, the act of subsequently formulating them does render them not “purified” within the context of an ingredient list.

In one embodiment, the compounds disclosed herein are purified by extracting the soluble compounds from plant material with ethanol, e.g., a cannabinoid, a terpene, etc. In one embodiment, the compounds disclosed herein are purified by chromatography techniques, such as supercritical fluid chromatography, e.g., a cannabinoid, a terpene, etc.

In one embodiment, the one or more purified cannabinoids are chosen from THC, Δ9-THC, Δ8-THC, THCA, THCV, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

In one embodiment, an Active Ingredient is a terpene. In an embodiment, each of the First Composition and the second composition may individually comprise one or more terpenes. In an embodiment, only one of the First Composition or the Second Composition comprises terpenes. In an embodiment, only one of the First Composition or the Second Composition comprises terpenes, and it is only a single type of terpene (e.g. a first terpene). In an embodiment, each of the First Composition and the Second Composition comprise only a single terpene, and that terpene may be the same in both (e.g. a first terpene) or different (e.g., a first terpene in one and a second terpene in the other).

As used herein, the term “terpene” refers to a compound built on an isoprenoid structure or produced by combining isoprene units, 5 carbon structures. Terpenes are also associated with producing smell in plants where terpenes are part of a class of secondary compounds.

Within the context of this disclosure, the term “terpene” does not necessarily require 5 carbons or multiples of 5 carbons. It is understood that a reaction with isoprene units does not always result in a terpene comprising all the carbon atoms.

Within the context of this disclosure, the term “terpene” includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. As well as isomeric, enantiomeric, or optically active derivatives.

Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids.

Within the context of this disclosure, the term terpene includes the α-(alpha), β-(beta), γ-(gamma), oxo-, isomers, stereoisomers or any combinations thereof.

Examples of terpenes within the context of this disclosure include: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha, Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Caryophyllene (Beta-Caryophyllene), Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/lcosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

As used herein, the term “7,8-dihydro-alpha-ionone” refers to a compound with the following structural formula:

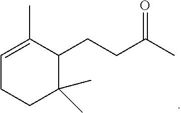

7,8-dihydro-alpha-ionone is often characterized as having a woody, floral odor.

As used herein, the term “7,8-dihydro-beta-ionone” refers to a compound with the following structural formula:

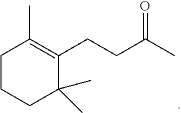

7,8-dihydro-beta-ionone is often characterized as having a woody aroma.

As used herein, the term “acetanisole” refers to a compound with the following structural formula:

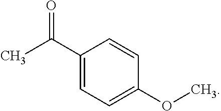

Acetanisole is often characterized as having a sweet, anisic, vanilla-like aroma with powdery, balsamic, and benzaldehyde nuances. Acetanisole is also used as a flavoring agent.

As used herein, the term “acetic acid” refers to a compound with the following structural formula:

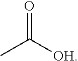

Acetic acid is often characterized as a one of the main constituents of vinegar.

As used herein, the term “acetyl cedrene” refers to a compound with the following structural formula:

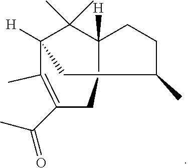

Acetyl cedrene is often characterized as having a warm, woody, amber musky aroma. Acetyl cedrene is also a fragrance agent.

As used herein, the term “anethole” refers to an aromatic compound having the following structural formula:

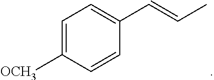

Anethole is often characterized as having a sweet, anise, licorice aroma. Anethole is used in a wide variety of fragrances and flavors.

As used herein, the term “anisole” refers to a compound with the following structural formula:

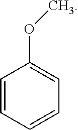

Anisole is often characterized as smelling like anise seeds.

As used herein, the term “benzaldehyde” refers to a compound with the following structural formula:

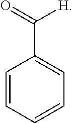

Benzaldehyde is often characterized as having an almond-like odor.

As used herein, the term “bergamotene” refers to a compound including either or both of alpha-cis-bergamotene and/or alpha-trans-Bergamotene in a pure and/or mixture of any ratio.

Alpha-cis-bergamotene refers to a compound with the following structural formula:

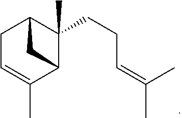

Alpha-cis-bergamotene is often characterized as having a strong odor of ground black pepper. Alpha-cis-bergamotene is an aroma component of many species of the family orchidaceae.

Alpha-trans-Bergamotene refers to the following structural formula:

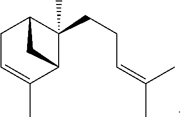

Alpha-trans-Bergamotene is often characterized as having a medium strength, warm, tea-leaf-like odor. Alpha-trans-Bergamotene is used in the chemical communication system of some species of aphids.

As used herein, the term “bisabolol” refers to a compound including either or both of alpha bisabolol and/or beta bisabolol in a pure and/or mixture of any ratio.

Alpha-bisabolol refers to a compound with the following structural formula:

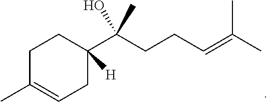 [SMILES: CC(C)=CCC[C@@](C)(O)[C@@H]1CC=C(C)CC1]

Alpha-bisabolol is often characterized as the active component of chamomile.

Beta-bisabolol refers to a compound with the following structural formula:

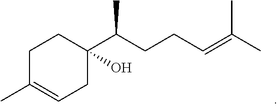 [SMILES: CC(C)=CCC[C@H](C)[C@@]1(O)CC=C(C)CC1]

Beta-bisabolol is often characterized as having a citrus, floral, lemon, sweet, herbaceous aroma.

As used herein, the term “borneol” refers to a compound having the following structural formula:

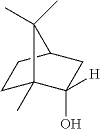

Borneol is often characterized as having a menthol aroma, similar to camphor. Borneol can also be converted into camphor.

As used herein, the term “bornyl acetate” refers to a compound with the following structural formula:

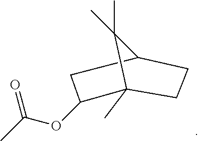

Bornyl acetate is often characterized as having a pine, camphoraceous, herbal, and balsamic odor.

As used herein, “butanoic/butyric acid” refers to a compound with the following structural formula:

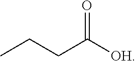

Butyric acid is often characterized as having an unpleasant, acrid odor normally found in perspiration, flatulence, and rancid butter.

As used herein, the term “cadinene” refers to a compound including either or both of alpha-cadinene and/or gamma-cadinene in a pure and/or mixture of any ratio.

Alpha-cadinene refers to a compound with the following structural formula:

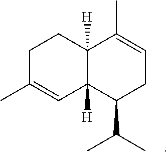

Alpha-cadinene is often characterized as having a pungent, smoky, woody, guaiac wood-like odor.

Gamma-cadinene refers to a compound having either of the following structural formula:

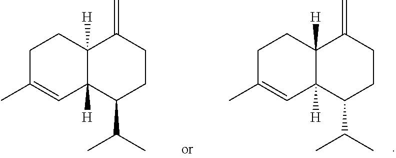

Gamma-cadinene is often characterized as having an herbaceous, herbal, woody aroma. Gamma-cadinene has shown some antimicrobial and antibacterial properties. Many species of termites and a few beetles utilize gamma-cadinene in their chemical communication systems.

As used herein, the term “cafestol” refers to a compound with the following structural formula:

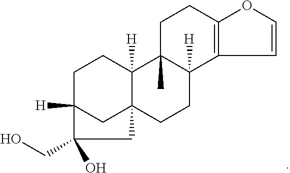

Cafestol is often characterized as major constituent in coffee.

As used herein, the term “caffeic acid” refers to a compound with the following structural formula:

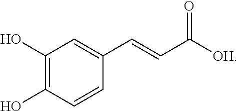 [SMILES: O=C(O)/C=C/c1ccc(O)c(O)c1]

Caffeic acid is often characterized as a key intermediate in the synthesis of lignin.

As used herein, the term “camphene” refers to a compound with the following structural formula:

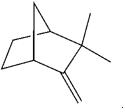

Camphene is often characterized as having a pungent, herbal, fir needle smell. Camphene is used in fragrances and food additives. Camphene is a minor constituent of many essential oils such as turpentine, cypress oil, camphor oil, citronella oil, neroli, ginger oil, and valerian.

As used herein, the term “camphor” refers to a compound with either of the following structural formulas:

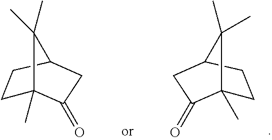

Within the context of this disclosure, the term “camphor” includes enantiomers as either a single form or a mixture in any ratio. Camphor has a very characteristic odor for which the tree is named. Camphor is used as a flavor and fragrance agent in chewing gum, hard candy, etc.

As used herein, the term “capsaicin” refers to a chemical compound with following structural formula:

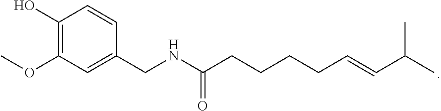 [SMILES: COc1cc(CNC(=O)CCCC/C=C/C(C)C)ccc1O]

Capsaicin is often odorless. Capsaicin is often characterized as an irritant for mammals, known for the sensation it provokes when inhaled, eaten, or applied to the skin.

As used herein, the term “carene” or “delta-3-carene” refers to a compound with the following structural formula:

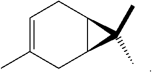

Delta-3-carene, sometimes referred to as “Carene”, is often characterized as having a sweet and pungent odor.

As used herein, the term “carotene” refers to any one of a series of related compounds having the chemical formula C₄₀Hₓ.

Within the context of this disclosure, the term “carotene” refers to any of the isomeric forms of carotene in a pure and/or mixture in any ratio. Carotene is often characterized as an important compound in the photosynthesis process.

As used herein, term “carvacrol” refers to a compound with the following structural formula:

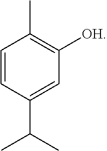

Carvacrol is often characterized as having a pungent odor of oregano. Carvacrol is often used as a flavor and fragrance agent and its flavor has been described as spicy, herbal, phenolic, medicinal, and woody.

As used herein, the term “dextro-carvone” refers to the S-(+) enantiomer of carvone with the following structural formula:

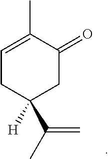

Dextro-carvone is often characterized as having a spicy, bready, caraway aroma. Dextro-carvone is found in mandarin peel oil and gingergrass oil. Dextro-carvone is the principal constituent of the oil from caraway seeds.

As used herein, the term “laevo-carvone” refers to the R-(−) enantiomer of carvone with the following structural formula:

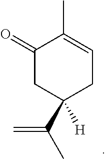

Laevo-carvone is often characterized as having a sweet, minty, herbaceous, spearmint odor. Laevo-carvone is found in spearmint and kuromoji oils.

As used herein, the term “beta-caryophyllene” refers to a compound with the following structural formula:

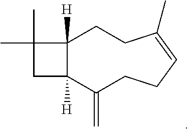

Beta-Caryophyllene is often characterized as a flavor component of black pepper. Beta-caryophyllene is often referred to as caryophyllene.

As used herein, the term “caryophyllene oxide” refers a compound with the following structural formula:

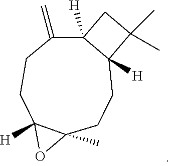 [SMILES: C=C1CC[C@H]2O[C@]2(C)CC[C@@H]2[C@@H]1CC2(C)C]

Caryophyllene oxide is often characterized as having a lemon balm odor.

As used herein, the term “cedrene” refers to either or both of alpha-cedrene and/or beta-Cedrene as pure forms or mixtures in any ratio.

Alpha-cedrene refers to a compound having the following structural formula:

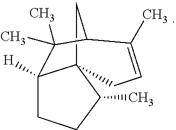

Alpha-cedrene is often characterized as having a medium strength, woody, sweet, fresh aroma of cedar. Alpha-cedrene is used in bakery items, sherbet and sorbet. Alpha-cedrene is a major component in the essential oil of cedar.

Beta-cedrene refers to a compound with the following structural formula:

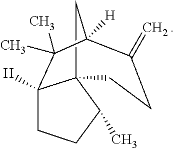

Beta-cedrene is often characterized as having a woody aroma of cedar. Beta-cedrene is a major component in the essential oil of cedar.

As used herein, the term “cedrene epoxide” or “alpha-cedrene epoxide” refers to a compound with the following structural formula:

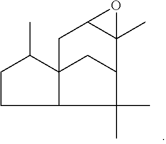 [SMILES: CC1CCC2C(C)(C)C3CC12CC1OC13C]

Alpha-cedrene epoxide is often characterized as having a woody, amber, tobacco, sandalwood, and fresh patchouli aroma. Alpha-cedrene epoxide is commonly used as a fragrance agent and a perfuming agent for cosmetics.

As used herein, the term “cedrol” refers to a compound with the following structural formula:

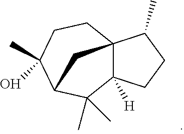 [SMILES: C[C@@H]1CC[C@H]2C(C)(C)[C@H]3C[C@@]12CC[C@@]3(C)O]

Cedrol is often characterized as having a very faint aroma that is sweet, cedar, and woody. Cedrol is a major component of cedar wood oil.

As used herein, the term “cembrene A” refers to a compound with the following structural formula:

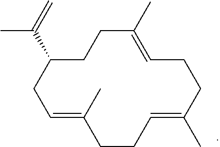 [SMILES: C=C(C)[C@H]1C/C=C(\C)CC/C=C(\C)CC/C=C(\C)CC1]

Cembrene A is often characterized as having a faint wax like odor.

As used herein, the term “chlorogenic acid” refers to a compound with the following structural formula:

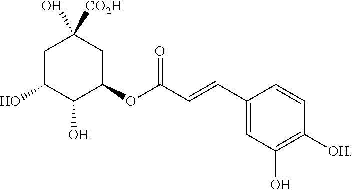 [SMILES: O=C(/C=C/c1ccc(O)c(O)c1)O[C@@H]1C[C@](O)(C(=O)O)C[C@@H](O)[C@H]1O]

Chlorogenic acid can often be found in peaches, prunes, and green coffee bean extract.

As used herein, the term “cinnamaldehyde” refers to a compound with the following structural formula:

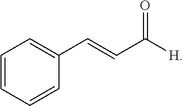

Cinnamaldehyde is often characterized as having a spicy, sweet cinnamon odor, and gives cinnamon its flavor. Cinnamaldehyde occurs naturally in the bark of cinnamon trees and other species of the genus Cinnamomum.

As used herein, the term “alpha-hexyl-cinnamaldehyde” refers to a compound with the following structural formula:

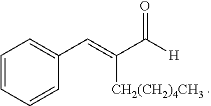

Alpha-hexyl-cinnamaldehyde is often characterized as having a sweet, floral, green, jasmine, citrus, and fruity aroma with powdery, tropical or spicy notes.

As used herein, the term “alpha-amyl-cinnamaldehyde” refers to a compound with the following structural formula:

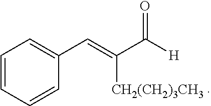

Alpha-amyl-cinnamaldehyde is often characterized as having a sweet, floral, fruity, herbal, jasmine, and tropical aroma.

As used herein, the term “cinnamic acid” refers to a compound with the following structural formula:

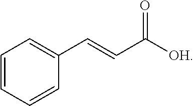 [SMILES: O=C(O)/C=C/c1ccccc1]

Cinnamic acid is often characterized as having a balsamic, sweet, storax, honey-like odor. Cinnamic acid is obtained from the oil of cinnamon or from balsams such as storax.

As used herein, the term “cinnamyl alcohol” refers to a compound with the following structural formula:

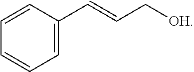

Cinnamyl alcohol is often characterized as having a cinnamon spice, floral green, and fermented odor with powdery balsamic nuances. As a flavor component cinnamyl alcohol has a green, floral, spicy, and honey flavor with a fermented yeasty nuance.

As used herein, the term “citronellal” refers to a compound with the following structural formula:

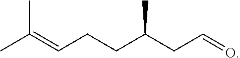

Citronellal is often characterized as making up to 80% of the leaf oil from Kaffir lime leaves and as the characteristic aroma.

As used herein, the term “citronellol” refers to either or both of the (+) and (−) enantiomers as pure forms or mixtures in any ratio.

The (+) enantiomer has the following structural formula:

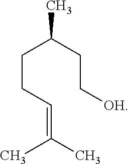

The (−) form has the following structural formula:

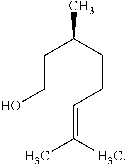

Citronellol is often characterized as having a floral, rosy, sweet, citrus with green, fatty, terpene-nuanced odor.

As used herein, the term “cryptone” refers to a compound with the following structural formula:

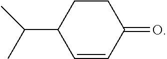

Cryptone is often characterized as having a woody minty herbaceous smell.

As used herein, the term “curcumene” refers either or both alpha-curcumene and/or gamma-curcumene as pure forms or mixtures in any ratio.

Alpha-curcumene has the following structural formula:

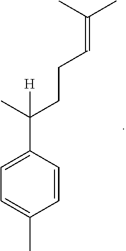

Alpha-curcumene is often characterized as having an odor similar to turmeric. Alpha-curcumene is found prominently in the Zingiber genus of ginger.

Gamma-curcumene has the following structural formula:

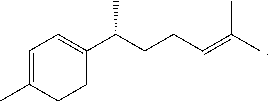

Gamma-curcumene is often characterized as having an earthy aroma. Gamma-curcumene is found prominently in the Libocedrus bidwillii tree.

As used herein, the term “decanal” refers to a compound with the following structural formula:

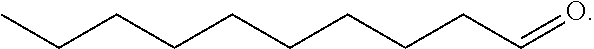

Decanal is often characterized as having a citrus odor. Decanal is used as a fragrance and flavoring agent.

As used herein, the term “dehydrovomifoliol” refers to a compound with the following structural formula:

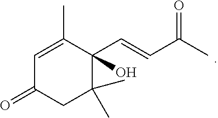 [SMILES: CC(=O)/C=C/[C@@]1(O)C(C)=CC(=O)CC1(C)C]

Dehydrovomifoliol is often characterized as having a fruity and flowery odor.

As used herein, the term “diallyl disulfide” refers to a compound with the following structural formula:

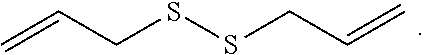

Diallyl disulfide is often characterized as having a strong, alliaceous, onion, and garlic-like odor.

As used herein, the term “dihydroactinidiolide” refers to a compound with the following structural formula:

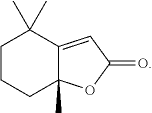

Dihydroactinidiolide is often characterized as having a fruity, musky, coumarin tea-like, peach aroma.

As used herein, the term “dimethyl disulfide” refers to a compound with the following structural formula:

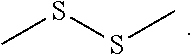

Dimethyl disulfide is often characterized as having a sulfurous, rotten aroma.

As used herein, the term “eicosane” or “icosane” refers to a compound with the chemical formula C₂₀H₄₂ and having 366,619 constitutional isomers with the following skeletal structure:

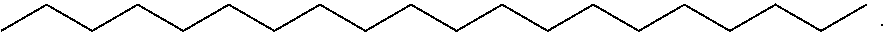 [SMILES: CCCCCCCCCCCCCCCCCCCC]

Eicosane is often characterized as having a waxy odor. Eicosane is used in fragrance concentrates.

As used herein, the term “beta-elemene” refers to a compound with the following

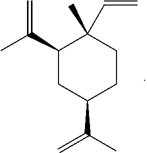

Beta-elemene is often characterized as having a sweet aroma.

As used herein, the term “estragole” refers to a compound with the following structural formula:

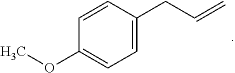

Estragole is an isomer of anethole. Estragole is often characterized as having an anise seed smell and occurs in tarragon oil, turpentine, and other essential oils.

As used herein, the term “ethyl acetate” refers to a compound with the following structural formula:

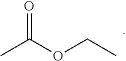

Ethyl acetate is often characterized as having an acidic, fruity, dirty, cheesy, fermented odor.

As used herein, the term “ethyl cinnamate” refers to a compound with the following structural formula:

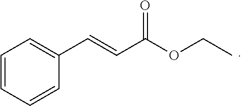

Ethyl Cinnamate is often characterized as having a sweet, balsamic, spicy, fruity, berry, and plum odor.

As used herein, the term “ethyl maltol” refers to a compound with the following

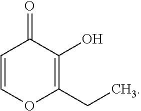

Ethyl maltol is often characterized as having a sweet smell similar to caramelized sugar and cooked fruit.

As used herein, the term “eucalyptol” refers to a terpene with the following structural formula:

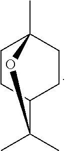

Eucalyptol is often characterized as having a mint-like smell. Eucalyptol is also known by 1,8-cineol, 1,8-cineole, cajeputol, 1,8-epoxy-p-menthane, 1,8-oxido-p-menthane, eucalyptol, eucalyptole, 1,3,3-trimethyl-2-oxabicyclo[2,2,2]octane, cineol, and cineole.

As used herein, the term “eudesmol” refers to alpha-eudesmol, beta-eudesmol, or gamma-eudesmol as pure forms or mixtures in any ratio.

Alpha-eudesmol has the following structural formula:

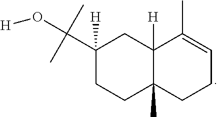 [SMILES: CC1=CCC[C@]2(C)CC[C@@H](C(C)(C)O)CC12]

Alpha-eudesmol is often characterized as having a sweet, woody odor.

Beta-eudesmol has the following structural formula:

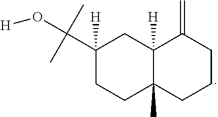 [SMILES: C=C1CCC[C@]2(C)CC[C@@H](C(C)(C)O)C[C@@H]12]

Beta-eudesmol is often characterized as having a sweet, green, woody, yuzu-like aroma.

Gamma-eudesmol has the following structural formula:

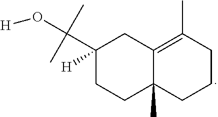 [SMILES: CC1=C2C[C@H](C(C)(C)O)CC[C@@]2(C)CCC1]

Gamma-eudesmol is often characterized as having a waxy, sweet, woody, floral odor.

As used herein the term “eugenol” refers to a compound with the following structural formula:

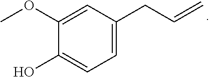

Eugenol is often characterized as causing the aromatic smell of cloves. Eugenol is found in insect attractants as well as UV absorbers.

As used herein, the term “euphol” refers to a compound with the following

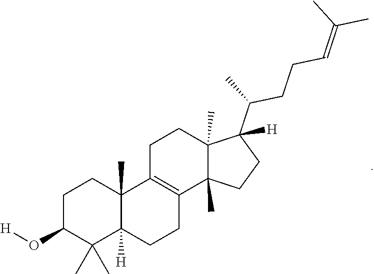 [SMILES: CC(C)=CCC[C@@H](C)[C@@H]1CC[C@]2(C)C3=C(CC[C@@]12C)[C@@]1(C)CC[C@H](O)C(C)(C)[C@@H]1CC3]

Euphol is often characterized as the main constituent in the sap of Euphorbia tirucalli.

As used herein, the term “farnesene” refers to six closely related compounds. (E, E)-alpha-farnesene is one of these six molecules and has the following structural formula:

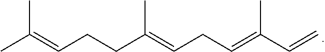

Within the context of this disclosure, the term “farnesene” refers to any one of the six closely related compounds, either alone or in combination of any other of those six closely related compounds. Farnesene is often characterized as having a fragrance of magnolia flowers and has citrus notes with green, woody, vegetative odor with hints of lavender.

As used herein, the term “farnesol” refers to a compound with the following structural formula:

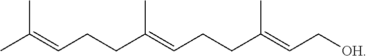 [SMILES: CC(C)=CCC/C(C)=C/CC/C(C)=C/CO]

Farnesol is often characterized as having a mild, fresh, sweet, floral, linden tree odor. Farnesol is used in cosmetics, flavors and fragrances.

As used herein, the term “fenchol” or “beta-fenchol” refers to a compound with the following structural formula:

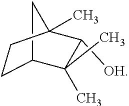

Beta-fenchol is often characterized as having a camphorous, piney, woody, dry, sweet, lemon scent. Beta-fenchol is used as a flavor and fragrance agent.

As used herein, the term “fenchone” refers to a compound with the following structural formula:

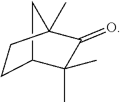

Fenchone is often characterized as having a camphorous, thuja, cedar leaf, herbal, earthy, woody aroma.

As used herein, the term “geraniol” refers to a compound of the following structural formula:

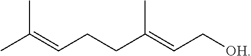

Geraniol is often characterized as having a sweet-rose like scent.

As used herein, the term “geranyl acetate” refers to a compound with the following structural formula:

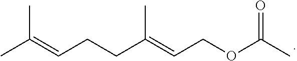

Geranyl acetate is often characterized as having a floral and fruit aroma. Geranyl acetate is found in a variety of natural oils from plants such as citronella, lemon grass, sassafras, rose, and many others.

As used herein, the term “geranylfarnesol” refers to a compound with the following structural formula:

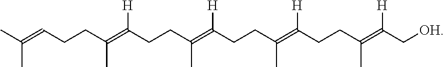 [SMILES: CC(C)=CCC/C(C)=C/CC/C(C)=C/CC/C(C)=C/CC/C(C)=C/CO]

Geranylfarnesol is often characterized as a clear colorless liquid.

As used herein, the term “germacrene” refers to a class of hydrocarbons. Within the context of this disclosure, the term “germacrene” refers to any of the five isomers as either pure forms or in any combination of the five isomers.

Germacrene A refers to the following structural formula:

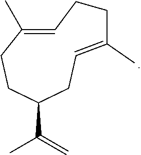

Germacrene B refers to the following structural formula:

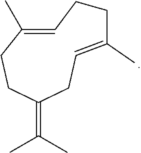

Germacrene C refers to the following structural formula:

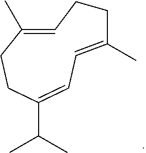

Germacrene D refers to the following structural formula:

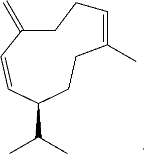

Germacrene E refers to the following structural formula:

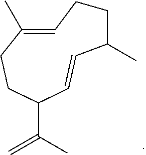

As used herein, the term “guaia-1(10), 11-diene” refers to a compound with the following structural formula:

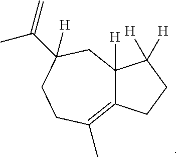

Guaia-1(10),11-diene is often characterized as having a sweet, woody aroma. Guaia-1(10),11-diene is used as a fragrance for a wide range of products from food additives, tobacco flavorings and general cosmetics, to odorizing rooms.

As used herein, the term “guaiacol” refers to a compound with the following structural formula:

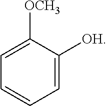

Guaiacol is often characterized as having a smoke-like, spicy, woody, medicinal odor. Guaiacol has a sweet, powdery, musty, vanilla, floral, almond flavor.

As used herein, the term “alpha-guaiene” refers to a compound with the following structural formula:

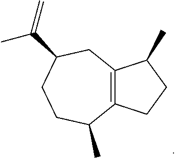

Alpha-guaiene is often characterized as having a sweet, earthy, woody, balsamic, peppery aroma.

As used herein, the term “gurjunene” or “alpha-gurjunene” refers to a compound with the following structural formula:

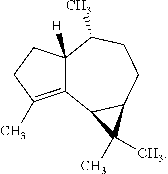

Alpha-gurjunene is often characterized as having a woody, balsamic odor. Alpha-gurjunene is used in cosmetics and fragrances.

As used herein, the term “herniarin” refers to a compound with the following structural formula:

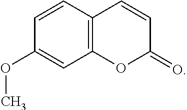

Herniarin is often found in Herniaria glabra, Ayanpana triplinervis, and in species of the genus Prunus (P. mahaleb, P. pensylvanica, and P. maximowiczil).

As used herein, the term “hexanaldehyde” refers to a compound with the following structural formula:

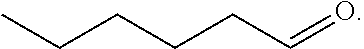

Hexanaldehyde is often characterized as having a grassy odor. Hexanaldehyde is also called hexanal.

As used herein, the term “hexanoic acid” refers to a compound with the following structural formula:

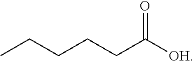

Hexanoic acid is often characterized as having a pungent, acrid, sour, fatty, sweaty, rancid cheese odor.

As used herein, the term “alpha-humulene” refers to a compound with the following structural formula:

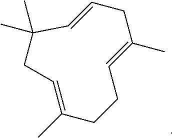

Alpha-humulene is often characterized as contributing to the flavor profile of beer. Alpha-humulene is sometimes often referred to as simply “Humulene” or “Caryophyllene” and in the context of this disclosure both may be used interchangeably.

As used herein, the term “beta-humulene” refers to a compound with the following structural formula:

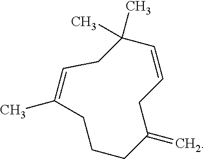

Beta-humulene is often characterized as having a green aroma.

As used herein, the term “ionol” refers to a compound with the following structural formula:

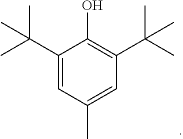 [SMILES: Cc1cc(C(C)(C)C)c(O)c(C(C)(C)C)c1]

Ionol is also known as Butylated Hydroxy Toluene (BHT). For the purpose of this disclosure “ionol” also refers to 3-oxo-alpha-ionol and/or beta-ionol in a pure and/or mixture of any ratio. Ionol is often characterized as having a sweet, woody, herbal, fruity, floral, tropical, and berry aroma.

As used herein, the term “ionone” refers to either or both alpha-ionone and/or beta-ionone as pure forms or mixtures in any ratio.

Alpha-ionone refers to the following structural formula:

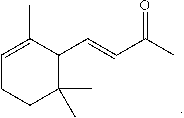

Beta-ionone refers to the following structural formula:

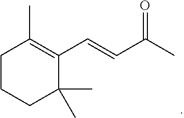

Alpha-ionone and beta-ionone as a mixture have an aroma similar to the violet flower.

As used herein, the term “ipsdienol” refers to a compound with the following structural formula:

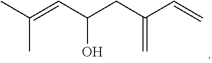

Ipsdienol is often characterized as having a balsamic, piney aroma.

As used herein, the term “isoamyl acetate” refers to a compound with the following structural formula:

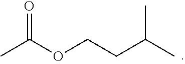

Isoamyl acetate is often characterized as having an odor similar to banana and pears.

As used herein, the term “isoamyl alcohol” refers to a compound with the following

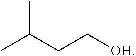

Isoamyl alcohol is often characterized as having a pungent, fermented, fusel, alcohol-like, aroma with fruity, banana and molasses notes. Isoamyl alcohol is used as a flavor and fragrance agent.

As used herein, the term “isoamyl formate” refers to a compound with the following structural formula:

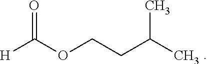

Isoamyl formate is often characterized as having a vinous, dry, earthy, fruity, green, plum, and blackcurrant aroma.

As used herein, the term “isoborneol” refers to a compound with the following structural formula:

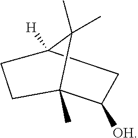

Isoborneol is often characterized as having a camphoraceous, sweet & musty aroma.

As used herein, the term “isomyrcenol” refers to a compound with the following structural formula:

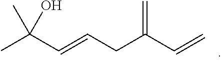

Isomyrcenol is often characterized as having a fresh, floral, lime-like odor. Isomyrcenol is used in the chemical communication systems of beetles.

As used herein, the term “isopulegol” refers to a compound with the following structural formula:

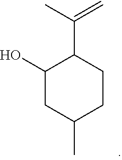

Isopulegol is often characterized as having a medium strength odor that is minty, cooling and bittersweet. Within the context of this disclosure, “isopulegol” may also refer to any number of isomeric forms.

As used herein, the term “isovaleric acid” refers to a compound with the following structural formula:

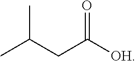

Isovaleric acid is often characterized as having a strong, pungent, sour, and sweaty cheese smell. Isovaleric acid is also known as 3-methylbutanoic acid.

As used herein, the term “isoprene” refers to a compound with the following structural formula:

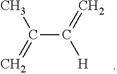

Isoprene is often considered as a building block for many other terpenes.

As used herein, the term “kahweol” refers to a compound with the following

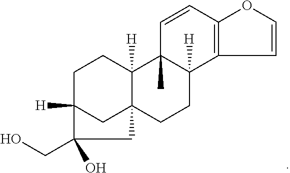

As used herein, the term “lavandulol” refers to either the R and S enantiomers as pure forms or mixture in any ratio with the following structural formula:

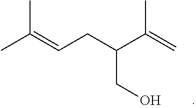

Lavandulol is often found in the essential oil of lavender.

As used herein, the term “limonene” refers to a terpene with the following structural formula:

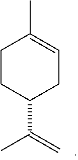

Limonene is often characterized as having a smell similar to oranges and other citrus fruits. Within the context of this disclosure, the term “Limonene” encompasses all possible enantiomers and isomers of the compound in as either individual compounds or in a racemic mixture.

As used herein, the term “gamma-linolenic acid” refers to a compound with the following structural formula:

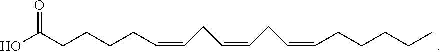 [SMILES: CCCCC/C=C\C/C=C\C/C=C\CCCCC(=O)O]

Gamma-linolenic acid is often characterized as an unsaturated fatty acid found primarily in vegetable oils.

As used herein, the term “linalool” refers to a terpene with the following structural formula:

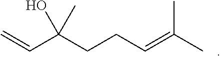

Linalool has two known enantiomeric forms. (S)-(+)-Linalool is often characterized as sweet and floral and the (R)-form is more woody and lavender-like. Within the context of this disclosure, the “Linalool” refers to either of the enantiomers or a racemic mixture of the two.

As used herein, the term “longifolene” refers to a compound having both (+) and (−) enantiomers. The (+) enantiomer refers to the following structural formula:

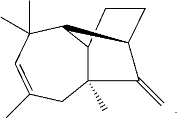

Within the context of this disclosure, the term “longifolene” refers to either of its (+) and/or (−) enantiomers in a pure form or mixture in any ratio. Longifolene is often characterized as having a sweet, woody, rosy, medical, fir needle odor.

As used herein, the term “alpha-longipinene” refers to a compound with the following structural formula:

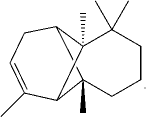 [SMILES: CC1=CCC2[C@]3(C)CCCC(C)(C)[C@@]2(C)C13]

Alpha-longipinene is often characterized as having a pine aroma.

As used herein, the term “lycopene” refers to a compound with the following

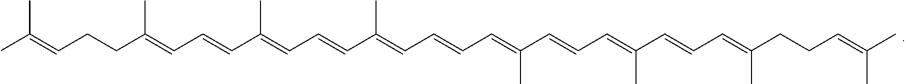 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

Lycopene is often characterized for its red color.

As used herein, the term “luteolin” refers to a compound with the structural formula:

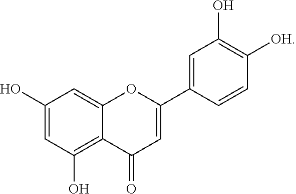 [SMILES: O=c1cc(-c2ccc(O)c(O)c2)oc2cc(O)cc(O)c12]

Luteolin is often found in leaves, rinds, barks, clover blossoms, and ragweed pollen.

As used herein, the term “menthol” refers to a compound with the following structural formula:

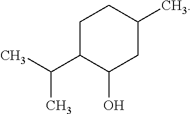

Menthol is often characterized as having a cooling, minty, peppermint aroma and flavor. Menthol is obtained from cornmint, peppermint or other mint oils.

As used herein, the term “methyl butyrate” refers to a compound with the following structural formula:

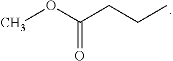

Methyl butyrate is often characterized as having a sulfurous odor.

As used herein, the term “3-Mercapto-2-Methylpentanal” refers to a compound with the following structural formula:

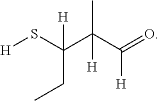

3-Mercapto-2-Methylpentanal is often characterized as having a sulfurous, alliaceous, garlic odor.

As used herein, the term “3-Mercapto-2-Methylpentanol” refers to a compound with the following structural formula:

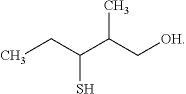

3-Mercapto-2-Methylpentanol is often characterized as having a very strong, sulfurous, onion type aroma.

As used herein, the term “mercaptan” refers to an organosulfur compound containing a carbon-bonded sulfhydryl. Mercaptan is often characterized as the main odor constituent added to assist in the detection of natural gas (which in pure form is odorless), and the “smell of natural gas” is due to the smell of the mercaptan thiol used as the odorant. Mercaptan is also referred to as thiol.

As used herein, the term “beta-mercaptoethanol” refers to a compound with the following structural formula:

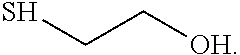

Beta-mercaptoethanol is often characterized as having an extremely strong and persistent aroma of sulfur.

As used herein, the term “mercaptoacetic acid” refers to a compound with the following structural formula:

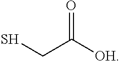

Mercaptoacetic acid is often characterized as having an ugly, sharp, acrid, skunk smell with plenty of endurance. Mercaptoacetic acid is also referred to as thioglycolic acid.

As used herein, the term “allyl mercaptan” refers to a compound with the following structural formula:

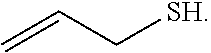

Allyl mercaptan is often characterized as having a strong sulfurous and alliaceous aroma.

As used herein, the term “benzyl mercaptan” refers to a compound with the following structural formula:

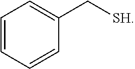

Benzyl mercaptan is often characterized as having a strong, sharp, alliaceous, sulfurous, onion, garlic, and horseradish aroma.

As used herein, the term “butyl mercaptan” refers to a compound with the following chemical formula C₄H₁₀S. In one embodiment, the butyl mercaptan has the following structural formula:

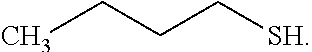

In one embodiment, the butyl mercaptan has the following structural formula:

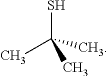

Butyl mercaptan is often characterized as having an extremely strong foul-smelling odor, commonly described as “skunk” odor.

As used herein, the term “methyl mercaptan” refers to a compound with the following structural formula:

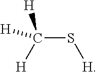

Methyl mercaptan is often characterized as having an extremely strong, sulfurous, decomposing cabbage, skunky aroma. Methyl mercaptan is also called methanethiol.

As used herein, the term “furfuryl mercaptan” refers to a compound with the following structural formula:

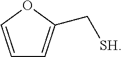

Furfuryl mercaptan is often characterized as having a very strong, oily, fatty, sulfurous, skunky odor.

As used herein, the term “ethylene mercaptan” refers to a compound with the following structural formula:

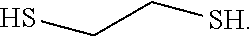

Ethylene mercaptan is often characterized as having a very high strength, sulfurous, skunky odor.

As used herein, the term “propyl mercaptan” refers to a compound with the following structural formula:

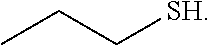

Propyl mercaptan is often characterized as having a high strength, alliaceous, natural gas, sweet onion, cabbage odor. Propyl mercaptan is also referred to as propanethiol.

As used herein, the term “thenyl mercaptan” refers to a compound with the following structural formula:

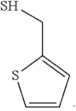

Thenyl mercaptan is often characterized as having a strong roast coffee, fishy aroma. Thenyl mercaptan is used as a flavoring agent mimicking the flavor of coffee.

As used herein, the term “methyl salicylate” refers to a compound with the following structural formula:

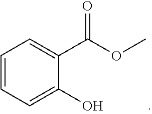

Methyl salicylate is often characterized as having a strong, distinct wintergreen aroma. Methyl salicylate is used as a fragrance agent, a food and beverage agent.

As used herein, the term “methylbutenol” refers to a compound with the following structural formula:

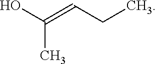

Methylbutenol is often characterized as having a very strong, malty herb aroma. Methylbutenol is released in high concentrations from lodgepole pine trees.

As used herein, the term “methyl-2-methylvalerate” refers to a carboxylic acid with the following structural formula:

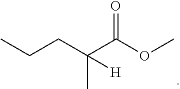

Methyl-2-methylvalerate is often characterized as having a fruity, sweet, berry, pineapple, apple, banana, green melon, and tropical aromas.

As used herein, the term “methyl thiobutyrate” refers to a compound with the following structural formula:

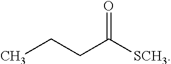

Methyl thiobutyrate is often characterized as a flavoring and food agent.

As used herein, the term “beta-myrcene” refers to a terpene with the following structural formula:

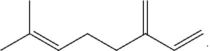

Beta-Myrcene is often characterized as having an earthy, fruity clove-like odor. Beta-myrcene is also referred to as “myrcene”.

As used herein, the term “gamma-muurolene” refers to a compound with the following structural formula:

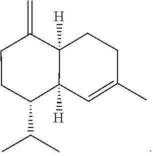

Gamma-muurolene is often characterized as having an herbal, woody, and spicy aroma.

As used herein, the term “nepetalactone” refers to a compound with the following structural formula:

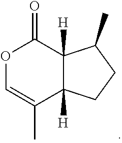

Nepetalactone is often characterized as having a valerian odor. Nepetalactone is also one of the main attractants and stimulants in catnip, which, when inhaled, insights wild play behavior in many cats.

As used herein, the term “nerol” refers to a compound with the following structural formula:

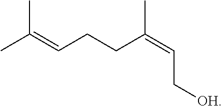

Nerol is often characterized a constituent of fragrances in cosmetics and as a flavor agent.

As used herein, the term “nerolidol” refers to a compound with either of the following structural formulas or any mixture thereby:

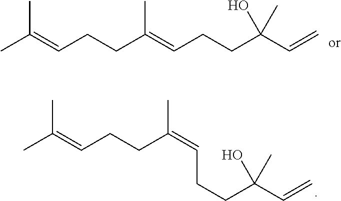

Nerolidol is often characterized as having a woody aroma, similar to fresh bark. Within the context of this disclosure, the term “Nerolidol” refers to either or both of the cis and trans isomers.

As used herein, the term “neryl acetate” refers to a compound with the following structural formula:

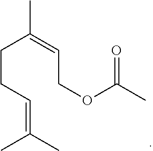

Neryl acetate is often characterized as having a floral and fruity aroma.

As used herein, the term “nonanaldehyde” refers to a compound with the following structural formula:

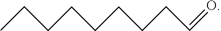

Nonanaldehyde is often characterized as having a soapy, citrus odor. Nonanaldehyde is a component of perfumes, although it also occurs in several natural oils. Nonanaldehyde is also referred to as nonanal.

As used herein, the term “nonanoic acid” refers to a carboxylic acid with the following structural formula:

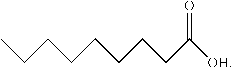

Nonanoic acid is often characterized as having an unpleasant, rancid, waxy, dirty, and cheesy aroma.

As used herein, the term “ocimene” refers to a compound with one of the isomeric forms:

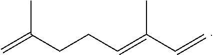

Within the context of this disclosure, ocimene refers to any of the isomers in a single pure form or a mixture in any ratio.

As used herein, the term “octanal” refers to a compound with the following

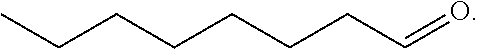

Octanal is often characterized as having a very strong waxy, citrus, orange peel aroma.

As used herein, the term “octanoic acid” refers to a compound with the following structural formula:

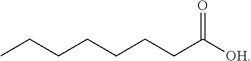

Octanoic acid is often characterized as having a musty, rancid, cheesy, sweat-like odor Octanoic acid is used as a cosmetic surfactant and as a flavoring agent for processed cheeses. Octanoic acid is also referred to as caprylic acid.

As used herein, the term “p-cymene” refers to a compound with the following structural formula:

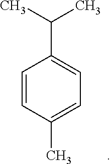

Within context of this disclosure, Cymene may refer to p-Cymene or any other isomeric form, e.g., m-Cymene or o-Cymene.

As used herein, the term “pentyl butyrate” refers to a compound with the following structural formula:

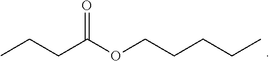

Pentyl butyrate is often characterized as having a smell reminiscent of pear or apricot.

As used herein, the term “phellandrene” refers to either of the two structural formulas:

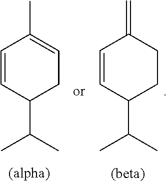

Alpha-phellandrene is often characterized as having a citrus, herbal, black pepper-like odor. Beta-phellandrene is often characterized as having a peppery-minty and slightly citrusy odor with a slight minty-turpentine note.

As used herein, the term “phenylacetaldehyde” refers to a compound with the following structural formula:

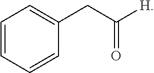

Phenylacetaldehyde is often characterized as having a strong pungent honey-like sweet and green floral odor. Phenylacetaldehyde is found in buckwheat, chocolate, and many other foods and flowers.

As used herein, the term “phenylethanethiol” refers to a compound with the following chemical formula: C₈H₁₀S comprising of multiple isomers.

Within the context of this disclosure, phenylethanethiol refers to any of the isomers as a pure compound and/or in any mixture. Phenylethanethiol is often characterized as having the high strength odor of rubber.

As used herein, the term “phenylacetic acid” refers to a compound with the following structural formula:

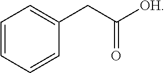

Phenylacetic acid is often characterized as having a sour yet sweet, waxy, civet, honey, rosey, floral odor similar to honeysuckle with notes of chocolate and tobacco.

Phenylacetic acid is also used in cosmetics for perfuming.

As used herein, the term “phytol” refers to a compound with the following

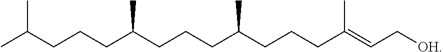 [SMILES: C/C(=C\CO)CCC[C@H](C)CCC[C@H](C)CCCC(C)C]

Phytol is often characterized having a mild floral, balsamic, and green tea type of aroma.

As used herein, the term “alpha-pinene” refers to a compound with either of the following structural formulas:

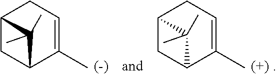

Alpha-Pinene is often characterized as having a pine tree like aroma.

As used herein, the term “beta-pinene” refers to a terpene with the following structural formula:

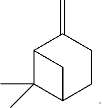

Beta-Pinene is often characterized as having a woody-green pine-like smell. Beta-Pinene is one of the most abundant compounds released by forest trees. Beta-Pinene is an isomer of pinene.

As used herein the term “pristimerin” refers to a compound with the following structural formula:

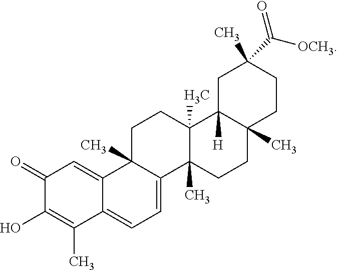

Pristimerin is often characterized as having antiviral and antitumor properties. Pristimerin is also called celastrol methyl ester celastrol.

As used herein, the term “pulegone” refers to a compound with the following structural formula:

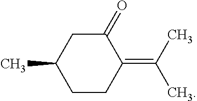

Pulegone is often characterized as having a smell similar to peppermint.

As used herein, the term “quercetin” refers to a compound with the following structural formula:

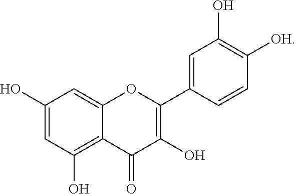 [SMILES: O=c1c(O)c(-c2ccc(O)c(O)c2)oc2cc(O)cc(O)c12]

As used herein, the term “retinol” refers to a compound with the following structural formula:

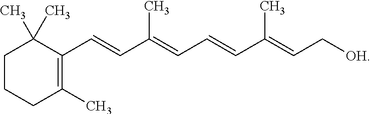 [SMILES: CC1=C(/C=C/C(C)=C/C=C/C(C)=C/CO)C(C)(C)CCC1]

Retinol is often characterized as a type of vitamin A that can be converted to other types of vitamin A.

As used herein, the term “rutin” refers to a compound with the following structural formula:

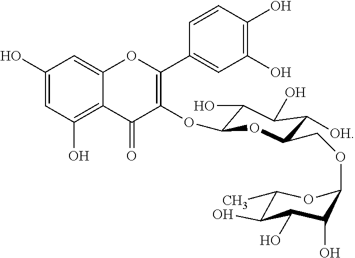

Rutin is often characterized as citrus flavonoid and found in many plants.

As used herein, the term “sabinene” refers to a compound with the following structural formula:

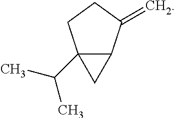

Sabinene is often characterized as having a peppery, woody, herbaceous, and spicy pine odor with citrus notes. Sabinene is found in oak trees, tea tree oil, black pepper and is a major constituent of carrot seed oil. Within the context of this disclosure, the term sabinene refers to either a enantiomeric form or a mixture in any ratio.

As used herein, the term “sabinene hydrate” refers to a compound with the following structural formula:

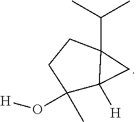

Sabinene hydrate is often characterized as having an herbal, minty, eucalyptol, and terpy odor with a spicy nuance.

As used herein, the term “cis-sabinene hydrate” refers to a compound with the following structural formula.

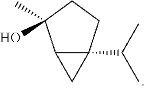

Cis-sabinene hydrate is often characterized as having a balsamic odor.

As used herein, the term “trans-sabinene hydrate” refers to the following structural formula.

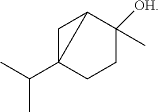

Trans-sabinene hydrate is often characterized as having a woody, balsamic odor.

As used herein, the term “safranal” refers to a compound the following structural formula:

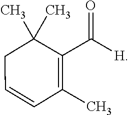

Safranal is often characterized as the component primarily responsible for the aroma of saffron.

As used herein, the term “alpha-selinene” refers to a compound with the following

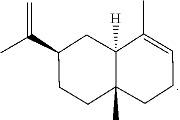

Alpha-selinene is often characterized as having a distinct odor of amber. Alpha-selinene is one of the principal components of the oil from celery seeds.

As used herein, the term “alpha-sinensal” refers to a compound with the following structural formula:

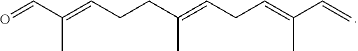 [SMILES: C=C/C(C)=C/C/C=C(\C)CC/C=C(\C)C=O]

Alpha-sinensal is often characterized as having a citrus orange, mandarin aroma.

As used herein, the term “beta-sinensal” refers to a compound with the following structural formula:

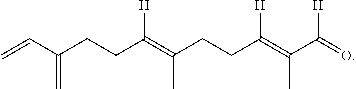 [SMILES: C=CC(=C)CC/C=C(\C)CC/C=C(\C)C=O]

Beta-sinensal is often characterized as having a sweet, fresh, waxy, and citrus odor. Beta-sinensal is used in fragrances.

As used herein, the term “beta-sitosterol” refers to a compound with the following structural formula:

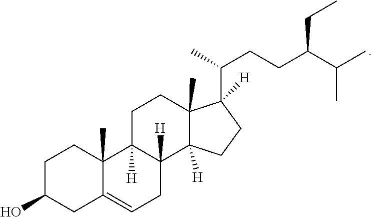

Beta-sitosterol is found in pecans, avocados, pumpkin seeds, cashews, corn oils, and soybeans.

As used herein, the term “squalene” refers to a compound with the following structural formula:

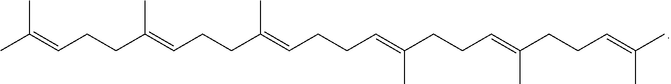 [SMILES: CC(C)=CCC/C(C)=C/CC/C(C)=C/CC/C=C(\C)CC/C=C(\C)CCC=C(C)C]

Squalene is often characterized as playing an important part in the synthesis of plant and animal sterols.

As used herein, the term “taxadiene” refers to a compound with the following structural formula:

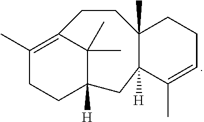 [SMILES: CC1=CCC[C@@]2(C)CCC3=C(C)CC[C@@H](C[C@H]12)C3(C)C]

Taxadiene is an intermediate in the synthesis of taxol.

As used herein, the term “terpin” refers to a compound of the following structural formula:

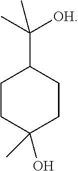

As used herein the term “terpineol” refers to a compound with the following structural formula:

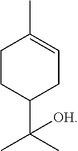

Within the context of this disclosure, terpineol includes any of the isomeric forms either as a single form or a mixture in any ratio. Terpineol is used extensively in the perfume industry. Within the context of this disclosure, terpineol refers to any and all isomeric forms, alpha, beta, gamma, etc, in any ratio or combination,

As used herein, the term “terpinen-4-ol” refers to the isomer of terpineol that has the following structural formula:

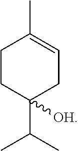

Terpinen-4-ol is often characterized as having an herbaceous, peppery, woody odor and is used in commercial fragrances. Terpinen-4-ol is considered the primary active ingredient of tea tree oil.

As used herein, the term “alpha-terpinene” refers a compound the following structural formula:

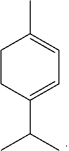

Alpha-terpinene is often characterized as having a lemony-citrus aroma. Alpha-terpinene has been isolated from cardamom and marjoram oils.

As used herein, the term “gamma-terpinene” refers to a compound with the following structural formula:

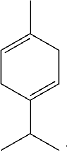

Gamma-terpinene is often characterized as having an herbaceous, citrusy sweet aroma.

As used herein, the term “terpinolene” or “delta-terpinene” refers to a compound with the following structural formula:

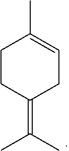

Terpinolene is often characterized as having an herbal aroma. Terpinolene's flavor has been described as sweet, woody, lemon, and lime-like.

As used herein, the term “thiophenol” refers to a compound with the following structural formula:

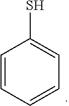

Thiophenol is often characterized as having a sulfurous odor.

As used herein, the term “thujone” refers to a compound with either of the following structural formulas:

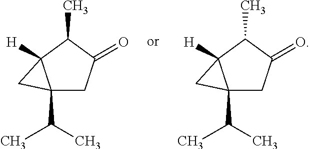

Within the context of this closure, the term “thujone” refers to any of the isomeric forms in a pure form or mixture of any ratio. Thujone is often characterized as having a menthol like odor.

As used herein, the term “thymol” refers to a compound with the following structural formula:

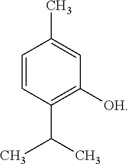

Thymol is often found in oil of thyme.

As used herein, the term “alpha-tocopherol” refers to a compound with the following structural formula:

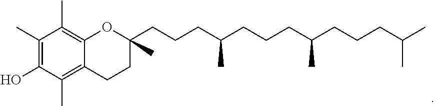 [SMILES: Cc1c(C)c2c(c(C)c1O)CC[C@@](C)(CCC[C@H](C)CCC[C@H](C)CCCC(C)C)O2]

Alpha-tocopherol is often characterized as having a very bland, slight, vegetable oil-like odor.

As used herein, the term “tonka undecanone” refers to a compound with the following structural formula:

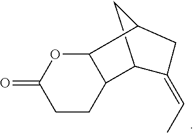

Tonka undecanone is often characterized as having a sweet, spicy, balsamic, and Tonka woody tobacco aroma.

As used herein, the term “undecanal” refers to an aldehyde that has the following structural formula:

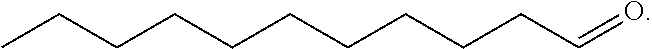

Undecanal is often characterized as having a soapy, aldehydic, waxy, and slightly effervescent orangey citrus-like odor with a watermelon, pineapple and cilantro background. Undecanal is a common component of perfumes.

As used herein, the term “valeraldehyde” or “pentanal” refers to a compound with the following structural formula:

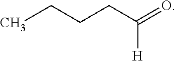

Valeraldehyde is often characterized as having a fermented type, diffusive, bready, fruity odor with berry nuances. Used as a flavoring agent, valeraldehyde has a wine-like, fermented, bready, cocoa flavor with chocolate notes.

As used herein, the term “verdoxan” refers to a cyclic ether with the following structural formula:

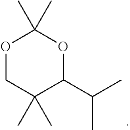

Verdoxan is often characterized as having a medium strength odor of earthy, fruity, green, herbal, sawn old wood. Verdoxan is used in fragrances and cosmetics.

As used herein, the term “alpha-ylangene” refers to a compound with the following structural formula:

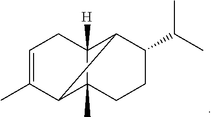

Alpha-ylangene is often characterized as having a spicy, fruity aroma. Alpha-ylangene is a natural substance often found in wine and is emitted by some plants.

As used herein, the term “umbelliferone” refers to a compound with the following structural formula:

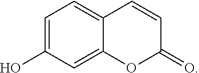 [SMILES: O=c1ccc2ccc(O)cc2o1]

As used herein, the term “vanillin” refers to a compound with the following structural formula:

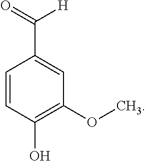 [SMILES: COc1cc(C=O)ccc1O]

Vanillin is often characterized as having a pleasant, sweet aroma, and the characteristic vanilla-like flavor.

In one embodiment, the one or more terpenes are chosen from Bornyl Acetate, Alpha-Bisabolol, Borneol, Camphene, Camphor, Carene, Beta-Caryophyllene, Cedrene, Cymene, Elemene, Eucalyptol, Eudesmol, Farnesene, Fenchol, Geraniol, Guaiacol, Humulene, Isoborneol, Limonene, Linalool, Menthol, Beta-Myrcene, Nerolidol, Ocimene, Phellandrene, Phytol, Pinene, Pulegone, Sabinene, Terpineol, Terpinolene, or Valencene.

In one embodiment, the one or more terpenes are purified.

In one embodiment, the one or more purified terpenes are chosen from Bornyl Acetate, Alpha-Bisabolol, Borneol, Camphene, Camphor, Carene, Beta-Caryophyllene, Cedrene, Cymene, Elemene, Eucalyptol, Eudesmol, Farnesene, Fenchol, Geraniol, Guaiacol, Humulene, Isoborneol, Limonene, Linalool, Menthol, Beta-Myrcene, Nerolidol, Ocimene, Phellandrene, Phytol, Pinene, Pulegone, Sabinene, Terpineol, Terpinolene, or Valencene.

In one embodiment, a First Composition comprises a First Active Ingredient and a Second Composition comprise a Second Active Ingredient.

In one embodiment, the First Active Ingredient and the Second Active Ingredient are the same.

In one embodiment, the First Active Ingredient and the Second Active Ingredient are the different.

In one embodiment, a First Active Ingredient is a cannabinoid and a Second Active Ingredient is a terpene.

In one embodiment, the devices disclosed herein comprise about a 1:1-20:1 molar ratio of a First Active Ingredient to a Second Active Ingredient.

In one embodiment, the devices disclosed herein comprise about a 1:1-15:1 molar ratio of a First Active Ingredient to a Second Active Ingredient.

In one embodiment, the devices disclosed herein comprise about a 1:1-10:1 molar ratio of a First Active Ingredient to a Second Active Ingredient.

In one embodiment, the devices disclosed herein comprise about a 1:1-5:1 molar ratio of a First Active Ingredient to a Second Active Ingredient.

In one embodiment, the devices disclosed herein comprise about a 1:1 molar ratio of a First Active Ingredient to a Second Active Ingredient.

As used herein, the term “molar ratio” refers to the proportion of a compound or compounds in relation to another compound or compounds as measured in moles. In one embodiment, the molar ratio is the proportion of a cannabinoid in relation to another cannabinoid. In one embodiment, the molar ratio is the proportion of a cannabinoid in relation to a terpene.

In one embodiment, the concentration of a First Active Ingredient is within 0-25% of the concentration of a Second Active Ingredient.

In one embodiment, the concentration of a First Active Ingredient is within 0-20% of the concentration of a Second Active Ingredient.

In one embodiment, the concentration of a First Active Ingredient is within 0-15% of the concentration of a Second Active Ingredient.

In one embodiment, the concentration of a First Active Ingredient is within 0-10% of the concentration of a Second Active Ingredient.

In one embodiment, the concentration of a First Active Ingredient is within 0-5% of the concentration of a Second Active Ingredient.

In one embodiment, at least one of a First Composition or a Second Composition comprises Vitamin E TPGS.

As used herein, the term “Vitamin E TPGS” refers to a product formed by the esterification of Vitamin E succinate with polyethylene glycol 1000 resulting in the following structural formula:

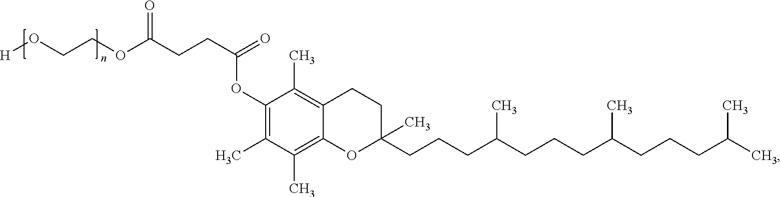

wherein “n” is an integer.

Within the context of this disclosure, Vitamin E TPGS may be formulated with compounds found in the cannabis plant to increase the solubility and bioavailability of poorly water soluble lipophilic compounds. In an embodiment, Vitamin E TPGS may be formulated with purified cannabinoids, such as those described herein, to increase the solubility and bioavailability of poorly water soluble lipophilic compounds

In one embodiment, the devices disclosed herein comprise about a 1:1 to 1:25 molar ratio of the concentration of a First Composition to the concentration of a Second Composition.

In one embodiment, the devices disclosed herein comprise about a 1:1 to 1:20 molar ratio of the concentration of a First Composition to the concentration of a Second Composition.

In one embodiment, the devices disclosed herein comprise about a 1:1 to 1:15 molar ratio of the concentration of a First Composition to the concentration of a Second Composition.

In one embodiment, the devices disclosed herein comprise about a 1:1 to 1:10 molar ratio of the concentration of a First Composition to the concentration of a Second Composition.

In one embodiment, the devices disclosed herein comprise about a 1:1 to 1:5 molar ratio of the concentration of a First Composition to the concentration of a Second Composition.

In one embodiment, the devices disclosed herein comprise about a 1:1 molar ratio of the concentration of a First Composition to the concentration of a Second Composition.

Within the context of this disclosure, the term “molar ratio of the concentration” refers to the proportion of moles regarding an Active Ingredient within a composition. In one example, the molar ratio of the concentration of a First Composition to the concentration of a Second Composition is of a purified cannabinoid in a First Composition and a purified terpene in a Second Composition.

In one embodiment, the devices disclosed herein comprise about 0.1-10 mass % of a First Composition.

In one embodiment, the devices disclosed herein comprise about 0.2-5.0 mass % of a First Composition.

In one embodiment, the devices disclosed herein comprise about 0.3-2.5 mass % of a First Composition.

In one embodiment, the devices disclosed herein comprise about 0.4-2.0 mass % of a First Composition.

In one embodiment, the devices disclosed herein comprise about 0.5-1.5 mass % of a First Composition.

In one embodiment, the devices disclosed herein comprise about 0.1-10 mass % of a Second Composition.

In one embodiment, the devices disclosed herein comprise about 0.2-5.0 mass % of a Second Composition.

In one embodiment, the devices disclosed herein comprise about 0.3-2.5 mass % of a Second Composition.

In one embodiment, the devices disclosed herein comprise about 0.4-2.0 mass % of a Second Composition.

In one embodiment, the devices disclosed herein comprise about 0.5-1.5 mass % of a Second Composition.

In one embodiment, an Aqueous Acid is about 0.1-10 mass % of a First Composition.

In one embodiment, an Aqueous Acid is about 0.2-5.0 mass % of a First Composition.

In one embodiment, an Aqueous Acid is about 0.3-2.5 mass % of a First

Composition.

In one embodiment, an Aqueous Acid is about 0.4-2.0 mass % of a First Composition.

In one embodiment, an Aqueous Acid is about 0.5-1.5 mass % of a First Composition.

In one embodiment, an Aqueous Carbonate Base is about 0.1-10 mass % of a Second Composition.

In one embodiment, an Aqueous Carbonate Base is about 0.2-5.0 mass % of a Second Composition.

In one embodiment, an Aqueous Carbonate Base is about 0.3-2.5 mass % of a Second Composition.

In one embodiment, an Aqueous Carbonate Base is about 0.4-2.0 mass % of a Second Composition.

In one embodiment, an Aqueous Carbonate Base is about 0.5-1.5 mass % of a Second Composition.

As used herein, the term “mass percent”, aka “percent by mass”, “mass %”, etc., refers to the amount of a compound relative to the entire mass of a sample as a fraction of 100. In one embodiment, mass percent is calculated with the following formula for a compound of interest:

[(mass of compound of interest in grams)/(total mass of composition in grams)]×100

In one example, a composition weighs 100 g and comprises 8 g of a purified cannabinoid. The mass percent of the purified cannabinoid is 8%. As the skilled person will appreciate, “mass %” may also be represented as “% w/w”.

In one embodiment, a means for expelling the First Composition from the First Chamber comprises a First Tube attached to the Nozzle and extending into the First Chamber.

In one embodiment, a means for expelling the Second Composition from the Second Chamber comprises a Second Tube attached to the Nozzle and extending into the Second Chamber.

In one embodiment, a means for expelling the First Composition and the Second Composition from the First Chamber and Second Chamber, respectively, comprises a Tube attached to the Nozzle that splits (e.g. into a First and Second Tube) and is attached both to the First Chamber and the Second Chamber.

As used herein, the term “Tube” (First, Second or otherwise) refers to a hollow cylinder fluidly connecting a Nozzle and an interior space of the Container, Chamber and/or bag. In one embodiment, the Tube allows a solution to move from a Chamber or bag within the Container through the Nozzle for administration of the Composition. In one embodiment, an atomizer nozzle draws a solution from a Chamber or bag through a Tube and expels the solution from the Nozzle. In one embodiment, the Tube is composed of rubber. In one embodiment, the Tube is composed of polyvinyl chloride. In one embodiment, a First Tube connects to a Nozzle. In one embodiment, a Second Tube connects to a Nozzle. In one embodiment, a First Tube and a Second Tube connect to a Nozzle.

In an embodiment, the device disclosed herein comprises and is configured for the delivery of unique combinations of a first cannabinoid and a terpene. In an embodiment, the First Composition comprises a first cannabinoid and the Second Composition comprises a terpene. In an embodiment, one or both of the First Composition and the Second Composition comprise both a first cannabinoid and a terpene. In one embodiment, the combination of a first cannabinoid and a terpene provide a synergistic effect at a mammalian cellular receptor, such as a G protein coupled receptor. In one embodiment, the receptor is CB1. In one embodiment, the receptor is CB2. In one embodiment, the receptor is GPR55. In one embodiment, the receptor is 5HT-1A. In one embodiment, the receptor is 5HT-2A. In one embodiment, the receptor is TRPV1. In one embodiment, the receptor is an opioid receptor, e.g., μ-opioid receptor.

In an embodiment, the device as disclosed herein comprises (i) a first cannabinoid chosen from THC, Δ9-THC, Δ8-THC, THCA, THCV, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA; and (ii) a terpene chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol, wherein the first cannabinoid and the terpene are in different compositions (First and Second Compositions) or are in the same composition. In an embodiment, the first cannabinoid and/or the terpene are purified.

In one embodiment, the first cannabinoid is THC and the terpene is Limonene.

In one embodiment, the first cannabinoid is THC and the terpene is Nerolidol.

In one embodiment, the first cannabinoid is THC and the terpene is Beta-Myrcene.

In one embodiment, the first cannabinoid is THC and the terpene is Linalool.

In one embodiment, the first cannabinoid is THC and the terpene is Alpha-Caryophyllene.

In one embodiment, the first cannabinoid is THC and the terpene is Beta-Caryophyllene.

In one embodiment, the first cannabinoid is THC and the terpene is Alpha-Pinene.

In one embodiment, the first cannabinoid is THC and the terpene is Beta-Pinene.

In one embodiment, the first cannabinoid is THC and the terpene is Alpha-Bisabolol.

In one embodiment, the first cannabinoid is THC and the terpene is Delta-3-Carene.

In one embodiment, the first cannabinoid is THC and the terpene is Borneol.

In one embodiment, the first cannabinoid is THC and the terpene is Eucalyptol.

In one embodiment, the first cannabinoid is THC and the terpene is p-Cymene.

In one embodiment, the first cannabinoid is THC and the terpene is Alpha-Terpineol.

In one embodiment, the first cannabinoid is THC and the terpene is Alpha-Humulene.

In one embodiment, the first cannabinoid is THC and the terpene is Terpinolene.

In one embodiment, the first cannabinoid is THC and the terpene is Pulegone.

In one embodiment, the first cannabinoid is THC and the terpene is Camphene.

In one embodiment, the first cannabinoid is THC and the terpene is Geraniol.

In one embodiment, the first cannabinoid is Δ9-THC and the terpene is Limonene.

In one embodiment, the first cannabinoid is Δ9-THC and the terpene is Nerolidol.

In one embodiment, the first cannabinoid is Δ9-THC and the terpene is Beta-Myrcene.

In one embodiment, the first cannabinoid is Δ9-THC and the terpene is Linalool.

In one embodiment, the first cannabinoid is Δ9-THC and the terpene is Alpha-Caryophyllene.

In one embodiment, the first cannabinoid is Δ9-THC and the terpene is Beta-Caryophyllene.

In one embodiment, the first cannabinoid is Δ9-THC and the terpene is Alpha-Pinene.

In one embodiment, the first cannabinoid is Δ9-THC and the terpene is Beta-Pinene.

In one embodiment, the first cannabinoid is Δ9-THC and the terpene is Alpha-Bisabolol.

In one embodiment, the first cannabinoid is Δ9-THC and the terpene is Delta-3-Carene.

In one embodiment, the first cannabinoid is Δ9-THC and the terpene is Borneol.

In one embodiment, the first cannabinoid is Δ9-THC and the terpene is Eucalyptol.

In one embodiment, the first cannabinoid is Δ9-THC and the terpene is p-Cymene.

In one embodiment, the first cannabinoid is Δ9-THC and the terpene is Alpha-Terpineol.

In one embodiment, the first cannabinoid is Δ9-THC and the terpene is Alpha-Humulene.

In one embodiment, the first cannabinoid is Δ9-THC and the terpene is

Terpinolene.

In one embodiment, the first cannabinoid is Δ9-THC and the terpene is Pulegone.

In one embodiment, the first cannabinoid is Δ9-THC and the terpene is Camphene.

In one embodiment, the first cannabinoid is Δ9-THC and the terpene is Geraniol.

In one embodiment, the first cannabinoid is Δ8-THC and the terpene is Limonene.

In one embodiment, the first cannabinoid is Δ8-THC and the terpene is Nerolidol.

In one embodiment, the first cannabinoid is Δ8-THC and the terpene is Beta-Myrcene.

In one embodiment, the first cannabinoid is Δ8-THC and the terpene is Linalool.

In one embodiment, the first cannabinoid is Δ8-THC and the terpene is Alpha-Caryophyllene.

In one embodiment, the first cannabinoid is Δ8-THC and the terpene is Beta-Caryophyllene.

In one embodiment, the first cannabinoid is Δ8-THC and the terpene is Alpha-Pinene.

In one embodiment, the first cannabinoid is Δ8-THC and the terpene is Beta-Pinene.

In one embodiment, the first cannabinoid is Δ8-THC and the terpene is Alpha-Bisabolol.

In one embodiment, the first cannabinoid is Δ8-THC and the terpene is Delta-3-Carene.

In one embodiment, the first cannabinoid is Δ8-THC and the terpene is Borneol.

In one embodiment, the first cannabinoid is Δ8-THC and the terpene is Eucalyptol.

In one embodiment, the first cannabinoid is Δ8-THC and the terpene is p-Cymene.

In one embodiment, the first cannabinoid is Δ8-THC and the terpene is Alpha-Terpineol.

In one embodiment, the first cannabinoid is Δ8-THC and the terpene is Alpha-Humulene.

In one embodiment, the first cannabinoid is Δ8-THC and the terpene is

Terpinolene.

In one embodiment, the first cannabinoid is Δ8-THC and the terpene is Pulegone.

In one embodiment, the first cannabinoid is Δ8-THC and the terpene is Camphene.

In one embodiment, the first cannabinoid is Δ8-THC and the terpene is Geraniol.

In one embodiment, the first cannabinoid is THCA and the terpene is Limonene.

In one embodiment, the first cannabinoid is THCA and the terpene is Nerolidol.

In one embodiment, the first cannabinoid is THCA and the terpene is Beta-Myrcene.

In one embodiment, the first cannabinoid is THCA and the terpene is Linalool.

In one embodiment, the first cannabinoid is THCA and the terpene is Alpha-Caryophyllene.

In one embodiment, the first cannabinoid is THCA and the terpene is Beta-Caryophyllene.

In one embodiment, the first cannabinoid is THCA and the terpene is Alpha-Pinene.

In one embodiment, the first cannabinoid is THCA and the terpene is Beta-Pinene.

In one embodiment, the first cannabinoid is THCA and the terpene is Alpha-Bisabolol.

In one embodiment, the first cannabinoid is THCA and the terpene is Delta-3-Carene.

In one embodiment, the first cannabinoid is THCA and the terpene is Borneol.

In one embodiment, the first cannabinoid is THCA and the terpene is Eucalyptol.

In one embodiment, the first cannabinoid is THCA and the terpene is p-Cymene.

In one embodiment, the first cannabinoid is THCA and the terpene is Alpha-Terpineol.

In one embodiment, the first cannabinoid is THCA and the terpene is Alpha-Humulene.

In one embodiment, the first cannabinoid is THCA and the terpene is Terpinolene.

In one embodiment, the first cannabinoid is THCA and the terpene is Pulegone.

In one embodiment, the first cannabinoid is THCA and the terpene is Camphene.

In one embodiment, the first cannabinoid is THCA and the terpene is Geraniol.

In one embodiment, the first cannabinoid is THCV and the terpene is Limonene.

In one embodiment, the first cannabinoid is THCV and the terpene is Nerolidol.

In one embodiment, the first cannabinoid is THCV and the terpene is Beta-Myrcene.

In one embodiment, the first cannabinoid is THCV and the terpene is Linalool.

In one embodiment, the first cannabinoid is THCV and the terpene is Alpha-Caryophyllene.

In one embodiment, the first cannabinoid is THCV and the terpene is Beta-Caryophyllene.

In one embodiment, the first cannabinoid is THCV and the terpene is Alpha-Pinene.

In one embodiment, the first cannabinoid is THCV and the terpene is Beta-Pinene.

In one embodiment, the first cannabinoid is THCV and the terpene is Alpha-Bisabolol.

In one embodiment, the first cannabinoid is THCV and the terpene is Delta-3-Carene.

In one embodiment, the first cannabinoid is THCV and the terpene is Borneol.

In one embodiment, the first cannabinoid is THCV and the terpene is Eucalyptol.

In one embodiment, the first cannabinoid is THCV and the terpene is p-Cymene.

In one embodiment, the first cannabinoid is THCV and the terpene is Alpha-Terpineol.

In one embodiment, the first cannabinoid is THCV and the terpene is Alpha-Humulene.

In one embodiment, the first cannabinoid is THCV and the terpene is Terpinolene.

In one embodiment, the first cannabinoid is THCV and the terpene is Pulegone.

In one embodiment, the first cannabinoid is THCV and the terpene is Camphene.

In one embodiment, the first cannabinoid is THCV and the terpene is Geraniol.

In one embodiment, the first cannabinoid is THCVA and the terpene is Limonene.

In one embodiment, the first cannabinoid is THCVA and the terpene is Nerolidol.

In one embodiment, the first cannabinoid is THCVA and the terpene is Beta-Myrcene.

In one embodiment, the first cannabinoid is THCVA and the terpene is Linalool.

In one embodiment, the first cannabinoid is THCVA and the terpene is Alpha-Caryophyllene.

In one embodiment, the first cannabinoid is THCVA and the terpene is Beta-Caryophyllene.

In one embodiment, the first cannabinoid is THCVA and the terpene is Alpha-Pinene.

In one embodiment, the first cannabinoid is THCVA and the terpene is Beta-Pinene.

In one embodiment, the first cannabinoid is THCVA and the terpene is Alpha-Bisabolol.

In one embodiment, the first cannabinoid is THCVA and the terpene is Delta-3-Carene.

In one embodiment, the first cannabinoid is THCVA and the terpene is Borneol.

In one embodiment, the first cannabinoid is THCVA and the terpene is Eucalyptol.

In one embodiment, the first cannabinoid is THCVA and the terpene is p-Cymene.

In one embodiment, the first cannabinoid is THCVA and the terpene is Alpha-Terpineol.

In one embodiment, the first cannabinoid is THCVA and the terpene is Alpha-Humulene.

In one embodiment, the first cannabinoid is THCVA and the terpene is Terpinolene.

In one embodiment, the first cannabinoid is THCVA and the terpene is Pulegone.

In one embodiment, the first cannabinoid is THCVA and the terpene is Camphene.

In one embodiment, the first cannabinoid is THCVA and the terpene is Geraniol.

In one embodiment, the first cannabinoid is CBD and the terpene is Limonene.

In one embodiment, the first cannabinoid is CBD and the terpene is Nerolidol.

In one embodiment, the first cannabinoid is CBD and the terpene is Beta-Myrcene.

In one embodiment, the first cannabinoid is CBD and the terpene is Linalool.

In one embodiment, the first cannabinoid is CBD and the terpene is Alpha-Caryophyllene.

In one embodiment, the first cannabinoid is CBD and the terpene is Beta-Caryophyllene.

In one embodiment, the first cannabinoid is CBD and the terpene is Alpha-Pinene.

In one embodiment, the first cannabinoid is CBD and the terpene is Beta-Pinene.

In one embodiment, the first cannabinoid is CBD and the terpene is Alpha-Bisabolol.

In one embodiment, the first cannabinoid is CBD and the terpene is Delta-3-Carene.

In one embodiment, the first cannabinoid is CBD and the terpene is Borneol.

In one embodiment, the first cannabinoid is CBD and the terpene is Eucalyptol.

In one embodiment, the first cannabinoid is CBD and the terpene is p-Cymene.

In one embodiment, the first cannabinoid is CBD and the terpene is Alpha-Terpineol.

In one embodiment, the first cannabinoid is CBD and the terpene is Alpha-Humulene.

In one embodiment, the first cannabinoid is CBD and the terpene is Terpinolene.

In one embodiment, the first cannabinoid is CBD and the terpene is Pulegone.

In one embodiment, the first cannabinoid is CBD and the terpene is Camphene.

In one embodiment, the first cannabinoid is CBD and the terpene is Geraniol.

In one embodiment, the first cannabinoid is CBDA and the terpene is Limonene.

In one embodiment, the first cannabinoid is CBDA and the terpene is Nerolidol.

In one embodiment, the first cannabinoid is CBDA and the terpene is Beta-Myrcene.

In one embodiment, the first cannabinoid is CBDA and the terpene is Linalool.

In one embodiment, the first cannabinoid is CBDA and the terpene is Alpha-Caryophyllene.

In one embodiment, the first cannabinoid is CBDA and the terpene is Beta-Caryophyllene.

In one embodiment, the first cannabinoid is CBDA and the terpene is Alpha-Pinene.

In one embodiment, the first cannabinoid is CBDA and the terpene is Beta-Pinene.

In one embodiment, the first cannabinoid is CBDA and the terpene is Alpha-Bisabolol.

In one embodiment, the first cannabinoid is CBDA and the terpene is Delta-3-Carene.

In one embodiment, the first cannabinoid is CBDA and the terpene is Borneol.

In one embodiment, the first cannabinoid is CBDA and the terpene is Eucalyptol.

In one embodiment, the first cannabinoid is CBDA and the terpene is p-Cymene.

In one embodiment, the first cannabinoid is CBDA and the terpene is Alpha-Terpineol.

In one embodiment, the first cannabinoid is CBDA and the terpene is Alpha-Humulene.

In one embodiment, the first cannabinoid is CBDA and the terpene is Terpinolene.

In one embodiment, the first cannabinoid is CBDA and the terpene is Pulegone.

In one embodiment, the first cannabinoid is CBDA and the terpene is Camphene.

In one embodiment, the first cannabinoid is CBDA and the terpene is Geraniol.

In one embodiment, the first cannabinoid is CBDV and the terpene is Limonene.

In one embodiment, the first cannabinoid is CBDV and the terpene is Nerolidol.

In one embodiment, the first cannabinoid is CBDV and the terpene is Beta-Myrcene.

In one embodiment, the first cannabinoid is CBDV and the terpene is Linalool.

In one embodiment, the first cannabinoid is CBDV and the terpene is Alpha-Caryophyllene.

In one embodiment, the first cannabinoid is CBDV and the terpene is Beta-Caryophyllene.

In one embodiment, the first cannabinoid is CBDV and the terpene is Alpha-Pinene.

In one embodiment, the first cannabinoid is CBDV and the terpene is Beta-Pinene.

In one embodiment, the first cannabinoid is CBDV and the terpene is Alpha-Bisabolol.

In one embodiment, the first cannabinoid is CBDV and the terpene is Delta-3-Carene.

In one embodiment, the first cannabinoid is CBDV and the terpene is Borneol.

In one embodiment, the first cannabinoid is CBDV and the terpene is Eucalyptol.

In one embodiment, the first cannabinoid is CBDV and the terpene is p-Cymene.

In one embodiment, the first cannabinoid is CBDV and the terpene is Alpha-Terpineol.

In one embodiment, the first cannabinoid is CBDV and the terpene is Alpha-Humulene.

In one embodiment, the first cannabinoid is CBDV and the terpene is Terpinolene.

In one embodiment, the first cannabinoid is CBDV and the terpene is Pulegone.

In one embodiment, the first cannabinoid is CBDV and the terpene is Camphene.

In one embodiment, the first cannabinoid is CBDV and the terpene is Geraniol.

In one embodiment, the first cannabinoid is CBDVA and the terpene is Limonene.

In one embodiment, the first cannabinoid is CBDVA and the terpene is Nerolidol.

In one embodiment, the first cannabinoid is CBDVA and the terpene is Beta-Myrcene.

In one embodiment, the first cannabinoid is CBDVA and the terpene is Linalool.

In one embodiment, the first cannabinoid is CBDVA and the terpene is Alpha-Caryophyllene.

In one embodiment, the first cannabinoid is CBDVA and the terpene is Beta-Caryophyllene.

In one embodiment, the first cannabinoid is CBDVA and the terpene is Alpha-Pinene.

In one embodiment, the first cannabinoid is CBDVA and the terpene is Beta-Pinene.

In one embodiment, the first cannabinoid is CBDVA and the terpene is Alpha-Bisabolol.

In one embodiment, the first cannabinoid is CBDVA and the terpene is Delta-3-Carene.

In one embodiment, the first cannabinoid is CBDVA and the terpene is Borneol.

In one embodiment, the first cannabinoid is CBDVA and the terpene is Eucalyptol.

In one embodiment, the first cannabinoid is CBDVA and the terpene is p-Cymene.

In one embodiment, the first cannabinoid is CBDVA and the terpene is Alpha-Terpineol.

In one embodiment, the first cannabinoid is CBDVA and the terpene is Alpha-Humulene.

In one embodiment, the first cannabinoid is CBDVA and the terpene is Terpinolene.

In one embodiment, the first cannabinoid is CBDVA and the terpene is Pulegone.

In one embodiment, the first cannabinoid is CBDVA and the terpene is Camphene.

In one embodiment, the first cannabinoid is CBDVA and the terpene is Geraniol.

In one embodiment, the first cannabinoid is CBC and the terpene is Limonene.

In one embodiment, the first cannabinoid is CBC and the terpene is Nerolidol.

In one embodiment, the first cannabinoid is CBC and the terpene is Beta-Myrcene.

In one embodiment, the first cannabinoid is CBC and the terpene is Linalool.

In one embodiment, the first cannabinoid is CBC and the terpene is Alpha-Caryophyllene.

In one embodiment, the first cannabinoid is CBC and the terpene is Beta-Caryophyllene.

In one embodiment, the first cannabinoid is CBC and the terpene is Alpha-Pinene.

In one embodiment, the first cannabinoid is CBC and the terpene is Beta-Pinene.

In one embodiment, the first cannabinoid is CBC and the terpene is Alpha-Bisabolol.

In one embodiment, the first cannabinoid is CBC and the terpene is Delta-3-Carene.

In one embodiment, the first cannabinoid is CBC and the terpene is Borneol.

In one embodiment, the first cannabinoid is CBC and the terpene is Eucalyptol.

In one embodiment, the first cannabinoid is CBC and the terpene is p-Cymene.

In one embodiment, the first cannabinoid is CBC and the terpene is Alpha-Terpineol.

In one embodiment, the first cannabinoid is CBC and the terpene is Alpha-Humulene.

In one embodiment, the first cannabinoid is CBC and the terpene is Terpinolene.

In one embodiment, the first cannabinoid is CBC and the terpene is Pulegone.

In one embodiment, the first cannabinoid is CBC and the terpene is Camphene.

In one embodiment, the first cannabinoid is CBC and the terpene is Geraniol.

In one embodiment, the first cannabinoid is CBCA and the terpene is Limonene.

In one embodiment, the first cannabinoid is CBCA and the terpene is Nerolidol.

In one embodiment, the first cannabinoid is CBCA and the terpene is Beta-Myrcene.

In one embodiment, the first cannabinoid is CBCA and the terpene is Linalool.

In one embodiment, the first cannabinoid is CBCA and the terpene is Alpha-Caryophyllene.

In one embodiment, the first cannabinoid is CBCA and the terpene is Beta-Caryophyllene.

In one embodiment, the first cannabinoid is CBCA and the terpene is Alpha-Pinene.

In one embodiment, the first cannabinoid is CBCA and the terpene is Beta-Pinene.

In one embodiment, the first cannabinoid is CBCA and the terpene is Alpha-Bisabolol.

In one embodiment, the first cannabinoid is CBCA and the terpene is Delta-3-Carene.

In one embodiment, the first cannabinoid is CBCA and the terpene is Borneol.

In one embodiment, the first cannabinoid is CBCA and the terpene is Eucalyptol.

In one embodiment, the first cannabinoid is CBCA and the terpene is p-Cymene.

In one embodiment, the first cannabinoid is CBCA and the terpene is Alpha-Terpineol.

In one embodiment, the first cannabinoid is CBCA and the terpene is Alpha-Humulene.

In one embodiment, the first cannabinoid is CBCA and the terpene is Terpinolene.

In one embodiment, the first cannabinoid is CBCA and the terpene is Pulegone.

In one embodiment, the first cannabinoid is CBCA and the terpene is Camphene.

In one embodiment, the first cannabinoid is CBCA and the terpene is Geraniol.

In one embodiment, the first cannabinoid is CBCV and the terpene is Limonene.

In one embodiment, the first cannabinoid is CBCV and the terpene is Nerolidol.

In one embodiment, the first cannabinoid is CBCV and the terpene is Beta-Myrcene.

In one embodiment, the first cannabinoid is CBCV and the terpene is Linalool.

In one embodiment, the first cannabinoid is CBCV and the terpene is Alpha-Caryophyllene.

In one embodiment, the first cannabinoid is CBCV and the terpene is Beta-Caryophyllene.

In one embodiment, the first cannabinoid is CBCV and the terpene is Alpha-Pinene.

In one embodiment, the first cannabinoid is CBCV and the terpene is Beta-Pinene.

In one embodiment, the first cannabinoid is CBCV and the terpene is Alpha-Bisabolol.

In one embodiment, the first cannabinoid is CBCV and the terpene is Delta-3-Carene.

In one embodiment, the first cannabinoid is CBCV and the terpene is Borneol.

In one embodiment, the first cannabinoid is CBCV and the terpene is Eucalyptol.

In one embodiment, the first cannabinoid is CBCV and the terpene is p-Cymene.

In one embodiment, the first cannabinoid is CBCV and the terpene is Alpha-Terpineol.

In one embodiment, the first cannabinoid is CBCV and the terpene is Alpha-Humulene.

In one embodiment, the first cannabinoid is CBCV and the terpene is Terpinolene.

In one embodiment, the first cannabinoid is CBCV and the terpene is Pulegone.

In one embodiment, the first cannabinoid is CBCV and the terpene is Camphene.

In one embodiment, the first cannabinoid is CBCV and the terpene is Geraniol.

In one embodiment, the first cannabinoid is CBCVA and the terpene is Limonene.

In one embodiment, the first cannabinoid is CBCVA and the terpene is Nerolidol.

In one embodiment, the first cannabinoid is CBCVA and the terpene is Beta-Myrcene.

In one embodiment, the first cannabinoid is CBCVA and the terpene is Linalool.

In one embodiment, the first cannabinoid is CBCVA and the terpene is Alpha-Caryophyllene.

In one embodiment, the first cannabinoid is CBCVA and the terpene is Beta-Caryophyllene.

In one embodiment, the first cannabinoid is CBCVA and the terpene is Alpha-Pinene.

In one embodiment, the first cannabinoid is CBCVA and the terpene is Beta-Pinene.

In one embodiment, the first cannabinoid is CBCVA and the terpene is Alpha-Bisabolol.

In one embodiment, the first cannabinoid is CBCVA and the terpene is Delta-3-Carene.

In one embodiment, the first cannabinoid is CBCVA and the terpene is Borneol.

In one embodiment, the first cannabinoid is CBCVA and the terpene is Eucalyptol.

In one embodiment, the first cannabinoid is CBCVA and the terpene is p-Cymene.

In one embodiment, the first cannabinoid is CBCVA and the terpene is Alpha-Terpineol.

In one embodiment, the first cannabinoid is CBCVA and the terpene is Alpha-Humulene.

In one embodiment, the first cannabinoid is CBCVA and the terpene is Terpinolene.

In one embodiment, the first cannabinoid is CBCVA and the terpene is Pulegone.

In one embodiment, the first cannabinoid is CBCVA and the terpene is Camphene.

In one embodiment, the first cannabinoid is CBCVA and the terpene is Geraniol.

In one embodiment, the first cannabinoid is CBG and the terpene is Limonene.

In one embodiment, the first cannabinoid is CBG and the terpene is Nerolidol.

In one embodiment, the first cannabinoid is CBG and the terpene is Beta-Myrcene.

In one embodiment, the first cannabinoid is CBG and the terpene is Linalool.

In one embodiment, the first cannabinoid is CBG and the terpene is Alpha-Caryophyllene.

In one embodiment, the first cannabinoid is CBG and the terpene is Beta-Caryophyllene.

In one embodiment, the first cannabinoid is CBG and the terpene is Alpha-Pinene.

In one embodiment, the first cannabinoid is CBG and the terpene is Beta-Pinene.

In one embodiment, the first cannabinoid is CBG and the terpene is Alpha-Bisabolol.

In one embodiment, the first cannabinoid is CBG and the terpene is Delta-3-Carene.

In one embodiment, the first cannabinoid is CBG and the terpene is Borneol.

In one embodiment, the first cannabinoid is CBG and the terpene is Eucalyptol.

In one embodiment, the first cannabinoid is CBG and the terpene is p-Cymene.

In one embodiment, the first cannabinoid is CBG and the terpene is Alpha-Terpineol.

In one embodiment, the first cannabinoid is CBG and the terpene is Alpha-Humulene.

In one embodiment, the first cannabinoid is CBG and the terpene is Terpinolene.

In one embodiment, the first cannabinoid is CBG and the terpene is Pulegone.

In one embodiment, the first cannabinoid is CBG and the terpene is Camphene.

In one embodiment, the first cannabinoid is CBG and the terpene is Geraniol.

In one embodiment, the first cannabinoid is CBGA and the terpene is Limonene.

In one embodiment, the first cannabinoid is CBGA and the terpene is Nerolidol.

In one embodiment, the first cannabinoid is CBGA and the terpene is Beta-Myrcene.

In one embodiment, the first cannabinoid is CBGA and the terpene is Linalool.

In one embodiment, the first cannabinoid is CBGA and the terpene is Alpha-Caryophyllene.

In one embodiment, the first cannabinoid is CBGA and the terpene is Beta-Caryophyllene.

In one embodiment, the first cannabinoid is CBGA and the terpene is Alpha-Pinene.

In one embodiment, the first cannabinoid is CBGA and the terpene is Beta-Pinene.

In one embodiment, the first cannabinoid is CBGA and the terpene is Alpha-Bisabolol.

In one embodiment, the first cannabinoid is CBGA and the terpene is Delta-3-Carene.

In one embodiment, the first cannabinoid is CBGA and the terpene is Borneol.

In one embodiment, the first cannabinoid is CBGA and the terpene is Eucalyptol.

In one embodiment, the first cannabinoid is CBGA and the terpene is p-Cymene.

In one embodiment, the first cannabinoid is CBGA and the terpene is Alpha-Terpineol.

In one embodiment, the first cannabinoid is CBGA and the terpene is Alpha-Humulene.

In one embodiment, the first cannabinoid is CBGA and the terpene is Terpinolene.

In one embodiment, the first cannabinoid is CBGA and the terpene is Pulegone.

In one embodiment, the first cannabinoid is CBGA and the terpene is Camphene.

In one embodiment, the first cannabinoid is CBGA and the terpene is Geraniol.

In one embodiment, the first cannabinoid is CBGV and the terpene is Limonene.

In one embodiment, the first cannabinoid is CBGV and the terpene is Nerolidol.

In one embodiment, the first cannabinoid is CBGV and the terpene is Beta-Myrcene.

In one embodiment, the first cannabinoid is CBGV and the terpene is Linalool.

In one embodiment, the first cannabinoid is CBGV and the terpene is Alpha-Caryophyllene.

In one embodiment, the first cannabinoid is CBGV and the terpene is Beta-Caryophyllene.

In one embodiment, the first cannabinoid is CBGV and the terpene is Alpha-Pinene.

In one embodiment, the first cannabinoid is CBGV and the terpene is Beta-Pinene.

In one embodiment, the first cannabinoid is CBGV and the terpene is Alpha-Bisabolol.

In one embodiment, the first cannabinoid is CBGV and the terpene is Delta-3-Carene.

In one embodiment, the first cannabinoid is CBGV and the terpene is Borneol.

In one embodiment, the first cannabinoid is CBGV and the terpene is Eucalyptol.

In one embodiment, the first cannabinoid is CBGV and the terpene is p-Cymene.

In one embodiment, the first cannabinoid is CBGV and the terpene is Alpha-Terpineol.

In one embodiment, the first cannabinoid is CBGV and the terpene is Alpha-Humulene.

In one embodiment, the first cannabinoid is CBGV and the terpene is Terpinolene.

In one embodiment, the first cannabinoid is CBGV and the terpene is Pulegone.

In one embodiment, the first cannabinoid is CBGV and the terpene is Camphene.

In one embodiment, the first cannabinoid is CBGV and the terpene is Geraniol.

In one embodiment, the first cannabinoid is CBGVA and the terpene is Limonene.

In one embodiment, the first cannabinoid is CBGVA and the terpene is Nerolidol.

In one embodiment, the first cannabinoid is CBGVA and the terpene is Beta-Myrcene.

In one embodiment, the first cannabinoid is CBGVA and the terpene is Linalool.

In one embodiment, the first cannabinoid is CBGVA and the terpene is Alpha-Caryophyllene.

In one embodiment, the first cannabinoid is CBGVA and the terpene is Beta-Caryophyllene.

In one embodiment, the first cannabinoid is CBGVA and the terpene is Alpha-Pinene.

In one embodiment, the first cannabinoid is CBGVA and the terpene is Beta-Pinene.

In one embodiment, the first cannabinoid is CBGVA and the terpene is Alpha-Bisabolol.

In one embodiment, the first cannabinoid is CBGVA and the terpene is Delta-3-Carene.

In one embodiment, the first cannabinoid is CBGVA and the terpene is Borneol.

In one embodiment, the first cannabinoid is CBGVA and the terpene is Eucalyptol.

In one embodiment, the first cannabinoid is CBGVA and the terpene is p-Cymene.

In one embodiment, the first cannabinoid is CBGVA and the terpene is Alpha-Terpineol.

In one embodiment, the first cannabinoid is CBGVA and the terpene is Alpha-Humulene.

In one embodiment, the first cannabinoid is CBGVA and the terpene is Terpinolene.

In one embodiment, the first cannabinoid is CBGVA and the terpene is Pulegone.

In one embodiment, the first cannabinoid is CBGVA and the terpene is Camphene.

In one embodiment, the first cannabinoid is CBGVA and the terpene is Geraniol.

In one embodiment, the first cannabinoid is CBN and the terpene is Limonene.

In one embodiment, the first cannabinoid is CBN and the terpene is Nerolidol.

In one embodiment, the first cannabinoid is CBN and the terpene is Beta-Myrcene.

In one embodiment, the first cannabinoid is CBN and the terpene is Linalool.

In one embodiment, the first cannabinoid is CBN and the terpene is Alpha-Caryophyllene.

In one embodiment, the first cannabinoid is CBN and the terpene is Beta-Caryophyllene.

In one embodiment, the first cannabinoid is CBN and the terpene is Alpha-Pinene.

In one embodiment, the first cannabinoid is CBN and the terpene is Beta-Pinene.

In one embodiment, the first cannabinoid is CBN and the terpene is Alpha-Bisabolol.

In one embodiment, the first cannabinoid is CBN and the terpene is Delta-3-Carene.

In one embodiment, the first cannabinoid is CBN and the terpene is Borneol.

In one embodiment, the first cannabinoid is CBN and the terpene is Eucalyptol.

In one embodiment, the first cannabinoid is CBN and the terpene is p-Cymene.

In one embodiment, the first cannabinoid is CBN and the terpene is Alpha-Terpineol.

In one embodiment, the first cannabinoid is CBN and the terpene is Alpha-Humulene.

In one embodiment, the first cannabinoid is CBN and the terpene is Terpinolene.

In one embodiment, the first cannabinoid is CBN and the terpene is Pulegone.

In one embodiment, the first cannabinoid is CBN and the terpene is Camphene.

In one embodiment, the first cannabinoid is CBN and the terpene is Geraniol.

In one embodiment, the first cannabinoid is CBNA and the terpene is Limonene.

In one embodiment, the first cannabinoid is CBNA and the terpene is Nerolidol.

In one embodiment, the first cannabinoid is CBNA and the terpene is Beta-Myrcene.

In one embodiment, the first cannabinoid is CBNA and the terpene is Linalool.

In one embodiment, the first cannabinoid is CBNA and the terpene is Alpha-Caryophyllene.

In one embodiment, the first cannabinoid is CBNA and the terpene is Beta-Caryophyllene.

In one embodiment, the first cannabinoid is CBNA and the terpene is Alpha-Pinene.

In one embodiment, the first cannabinoid is CBNA and the terpene is Beta-Pinene.

In one embodiment, the first cannabinoid is CBNA and the terpene is Alpha-Bisabolol.

In one embodiment, the first cannabinoid is CBNA and the terpene is Delta-3-Carene.

In one embodiment, the first cannabinoid is CBNA and the terpene is Borneol.

In one embodiment, the first cannabinoid is CBNA and the terpene is Eucalyptol.

In one embodiment, the first cannabinoid is CBNA and the terpene is p-Cymene.

In one embodiment, the first cannabinoid is CBNA and the terpene is Alpha-Terpineol.

In one embodiment, the first cannabinoid is CBNA and the terpene is Alpha-Humulene.

In one embodiment, the first cannabinoid is CBNA and the terpene is Terpinolene.

In one embodiment, the first cannabinoid is CBNA and the terpene is Pulegone.

In one embodiment, the first cannabinoid is CBNA and the terpene is Camphene.

In one embodiment, the first cannabinoid is CBNA and the terpene is Geraniol.

In one embodiment, the first cannabinoid is CBNV and the terpene is Limonene.

In one embodiment, the first cannabinoid is CBNV and the terpene is Nerolidol.

In one embodiment, the first cannabinoid is CBNV and the terpene is Beta-Myrcene.

In one embodiment, the first cannabinoid is CBNV and the terpene is Linalool.

In one embodiment, the first cannabinoid is CBNV and the terpene is Alpha-Caryophyllene.

In one embodiment, the first cannabinoid is CBNV and the terpene is Beta-Caryophyllene.

In one embodiment, the first cannabinoid is CBNV and the terpene is Alpha-Pinene.

In one embodiment, the first cannabinoid is CBNV and the terpene is Beta-Pinene.

In one embodiment, the first cannabinoid is CBNV and the terpene is Alpha-Bisabolol.

In one embodiment, the first cannabinoid is CBNV and the terpene is Delta-3-Carene.

In one embodiment, the first cannabinoid is CBNV and the terpene is Borneol.

In one embodiment, the first cannabinoid is CBNV and the terpene is Eucalyptol.

In one embodiment, the first cannabinoid is CBNV and the terpene is p-Cymene.

In one embodiment, the first cannabinoid is CBNV and the terpene is Alpha-Terpineol.

In one embodiment, the first cannabinoid is CBNV and the terpene is Alpha-Humulene.

In one embodiment, the first cannabinoid is CBNV and the terpene is Terpinolene.

In one embodiment, the first cannabinoid is CBNV and the terpene is Pulegone.

In one embodiment, the first cannabinoid is CBNV and the terpene is Camphene.

In one embodiment, the first cannabinoid is CBNV and the terpene is Geraniol.

In one embodiment, the first cannabinoid is CBNVA and the terpene is Limonene.

In one embodiment, the first cannabinoid is CBNVA and the terpene is Nerolidol.

In one embodiment, the first cannabinoid is CBNVA and the terpene is Beta-Myrcene.

In one embodiment, the first cannabinoid is CBNVA and the terpene is Linalool.

In one embodiment, the first cannabinoid is CBNVA and the terpene is Alpha-Caryophyllene.

In one embodiment, the first cannabinoid is CBNVA and the terpene is Beta-Caryophyllene.

In one embodiment, the first cannabinoid is CBNVA and the terpene is Alpha-Pinene.

In one embodiment, the first cannabinoid is CBNVA and the terpene is Beta-Pinene.

In one embodiment, the first cannabinoid is CBNVA and the terpene is Alpha-Bisabolol.

In one embodiment, the first cannabinoid is CBNVA and the terpene is Delta-3-Carene.

In one embodiment, the first cannabinoid is CBNVA and the terpene is Borneol.

In one embodiment, the first cannabinoid is CBNVA and the terpene is Eucalyptol.

In one embodiment, the first cannabinoid is CBNVA and the terpene is p-Cymene.

In one embodiment, the first cannabinoid is CBNVA and the terpene is Alpha-Terpineol.

In one embodiment, the first cannabinoid is CBNVA and the terpene is Alpha-Humulene.

In one embodiment, the first cannabinoid is CBNVA and the terpene is Terpinolene.

In one embodiment, the first cannabinoid is CBNVA and the terpene is Pulegone.

In one embodiment, the first cannabinoid is CBNVA and the terpene is Camphene.

In one embodiment, the first cannabinoid is CBNVA and the terpene is Geraniol.

In one embodiment, the first cannabinoid is CBND and the terpene is Limonene.

In one embodiment, the first cannabinoid is CBND and the terpene is Nerolidol.

In one embodiment, the first cannabinoid is CBND and the terpene is Beta-Myrcene.

In one embodiment, the first cannabinoid is CBND and the terpene is Linalool.

In one embodiment, the first cannabinoid is CBND and the terpene is Alpha-Caryophyllene.

In one embodiment, the first cannabinoid is CBND and the terpene is Beta-Caryophyllene.

In one embodiment, the first cannabinoid is CBND and the terpene is Alpha-Pinene.

In one embodiment, the first cannabinoid is CBND and the terpene is Beta-Pinene.

In one embodiment, the first cannabinoid is CBND and the terpene is Alpha-Bisabolol.

In one embodiment, the first cannabinoid is CBND and the terpene is Delta-3-Carene.

In one embodiment, the first cannabinoid is CBND and the terpene is Borneol.

In one embodiment, the first cannabinoid is CBND and the terpene is Eucalyptol.

In one embodiment, the first cannabinoid is CBND and the terpene is p-Cymene.

In one embodiment, the first cannabinoid is CBND and the terpene is Alpha-Terpineol.

In one embodiment, the first cannabinoid is CBND and the terpene is Alpha-Humulene.

In one embodiment, the first cannabinoid is CBND and the terpene is Terpinolene.

In one embodiment, the first cannabinoid is CBND and the terpene is Pulegone.

In one embodiment, the first cannabinoid is CBND and the terpene is Camphene.

In one embodiment, the first cannabinoid is CBND and the terpene is Geraniol.

In one embodiment, the first cannabinoid is CBNDA and the terpene is Limonene.

In one embodiment, the first cannabinoid is CBNDA and the terpene is Nerolidol.

In one embodiment, the first cannabinoid is CBNDA and the terpene is Beta-Myrcene.

In one embodiment, the first cannabinoid is CBNDA and the terpene is Linalool.

In one embodiment, the first cannabinoid is CBNDA and the terpene is Alpha-Caryophyllene.

In one embodiment, the first cannabinoid is CBNDA and the terpene is Beta-Caryophyllene.

In one embodiment, the first cannabinoid is CBNDA and the terpene is Alpha-Pinene.

In one embodiment, the first cannabinoid is CBNDA and the terpene is Beta-Pinene.

In one embodiment, the first cannabinoid is CBNDA and the terpene is Alpha-Bisabolol.

In one embodiment, the first cannabinoid is CBNDA and the terpene is Delta-3-Carene.

In one embodiment, the first cannabinoid is CBNDA and the terpene is Borneol.

In one embodiment, the first cannabinoid is CBNDA and the terpene is Eucalyptol.

In one embodiment, the first cannabinoid is CBNDA and the terpene is p-Cymene.

In one embodiment, the first cannabinoid is CBNDA and the terpene is Alpha-Terpineol.

In one embodiment, the first cannabinoid is CBNDA and the terpene is Alpha-Humulene.

In one embodiment, the first cannabinoid is CBNDA and the terpene is Terpinolene.

In one embodiment, the first cannabinoid is CBNDA and the terpene is Pulegone.

In one embodiment, the first cannabinoid is CBNDA and the terpene is Camphene.

In one embodiment, the first cannabinoid is CBNDA and the terpene is Geraniol.

In one embodiment, the first cannabinoid is CBNDV and the terpene is Limonene.

In one embodiment, the first cannabinoid is CBNDV and the terpene is Nerolidol.

In one embodiment, the first cannabinoid is CBNDV and the terpene is Beta-Myrcene.

In one embodiment, the first cannabinoid is CBNDV and the terpene is Linalool.

In one embodiment, the first cannabinoid is CBNDV and the terpene is Alpha-Caryophyllene.

In one embodiment, the first cannabinoid is CBNDV and the terpene is Beta-Caryophyllene.

In one embodiment, the first cannabinoid is CBNDV and the terpene is Alpha-Pinene.

In one embodiment, the first cannabinoid is CBNDV and the terpene is Beta-Pinene.

In one embodiment, the first cannabinoid is CBNDV and the terpene is Alpha-Bisabolol.

In one embodiment, the first cannabinoid is CBNDV and the terpene is Delta-3-Carene.

In one embodiment, the first cannabinoid is CBNDV and the terpene is Borneol.

In one embodiment, the first cannabinoid is CBNDV and the terpene is Eucalyptol.

In one embodiment, the first cannabinoid is CBNDV and the terpene is p-Cymene.

In one embodiment, the first cannabinoid is CBNDV and the terpene is Alpha-Terpineol.

In one embodiment, the first cannabinoid is CBNDV and the terpene is Alpha-Humulene.

In one embodiment, the first cannabinoid is CBNDV and the terpene is

Terpinolene.

In one embodiment, the first cannabinoid is CBNDV and the terpene is Pulegone.

In one embodiment, the first cannabinoid is CBNDV and the terpene is Camphene.

In one embodiment, the first cannabinoid is CBNDV and the terpene is Geraniol.

In one embodiment, the first cannabinoid is CBNDVA and the terpene is Limonene.

In one embodiment, the first cannabinoid is CBNDVA and the terpene is Nerolidol.

In one embodiment, the first cannabinoid is CBNDVA and the terpene is Beta-Myrcene.

In one embodiment, the first cannabinoid is CBNDVA and the terpene is Linalool.

In one embodiment, the first cannabinoid is CBNDVA and the terpene is Alpha-Caryophyllene.

In one embodiment, the first cannabinoid is CBNDVA and the terpene is Beta-Caryophyllene.

In one embodiment, the first cannabinoid is CBNDVA and the terpene is Alpha-Pinene.

In one embodiment, the first cannabinoid is CBNDVA and the terpene is Beta-Pinene.

In one embodiment, the first cannabinoid is CBNDVA and the terpene is Alpha-Bisabolol.

In one embodiment, the first cannabinoid is CBNDVA and the terpene is Delta-3-Carene.

In one embodiment, the first cannabinoid is CBNDVA and the terpene is Borneol.

In one embodiment, the first cannabinoid is CBNDVA and the terpene is Eucalyptol.

In one embodiment, the first cannabinoid is CBNDVA and the terpene is p-Cymene.

In one embodiment, the first cannabinoid is CBNDVA and the terpene is Alpha-Terpineol.

In one embodiment, the first cannabinoid is CBNDVA and the terpene is Alpha-Humulene.

In one embodiment, the first cannabinoid is CBNDVA and the terpene is Terpinolene.

In one embodiment, the first cannabinoid is CBNDVA and the terpene is Pulegone.

In one embodiment, the first cannabinoid is CBNDVA and the terpene is Camphene.

In one embodiment, the first cannabinoid is CBNDVA and the terpene is Geraniol.

In one embodiment, the first cannabinoid is CBL and the terpene is Limonene.

In one embodiment, the first cannabinoid is CBL and the terpene is Nerolidol.

In one embodiment, the first cannabinoid is CBL and the terpene is Beta-Myrcene.

In one embodiment, the first cannabinoid is CBL and the terpene is Linalool.

In one embodiment, the first cannabinoid is CBL and the terpene is Alpha-Caryophyllene.

In one embodiment, the first cannabinoid is CBL and the terpene is Beta-Caryophyllene.

In one embodiment, the first cannabinoid is CBL and the terpene is Alpha-Pinene.

In one embodiment, the first cannabinoid is CBL and the terpene is Beta-Pinene.

In one embodiment, the first cannabinoid is CBL and the terpene is Alpha-Bisabolol.

In one embodiment, the first cannabinoid is CBL and the terpene is Delta-3-Carene.

In one embodiment, the first cannabinoid is CBL and the terpene is Borneol.

In one embodiment, the first cannabinoid is CBL and the terpene is Eucalyptol.

In one embodiment, the first cannabinoid is CBL and the terpene is p-Cymene.

In one embodiment, the first cannabinoid is CBL and the terpene is Alpha-Terpineol.

In one embodiment, the first cannabinoid is CBL and the terpene is Alpha-Humulene.

In one embodiment, the first cannabinoid is CBL and the terpene is Terpinolene.

In one embodiment, the first cannabinoid is CBL and the terpene is Pulegone.

In one embodiment, the first cannabinoid is CBL and the terpene is Camphene.

In one embodiment, the first cannabinoid is CBL and the terpene is Geraniol.

In one embodiment, the first cannabinoid is CBLA and the terpene is Limonene.

In one embodiment, the first cannabinoid is CBLA and the terpene is Nerolidol.

In one embodiment, the first cannabinoid is CBLA and the terpene is Beta-Myrcene.

In one embodiment, the first cannabinoid is CBLA and the terpene is Linalool.

In one embodiment, the first cannabinoid is CBLA and the terpene is Alpha-Caryophyllene.

In one embodiment, the first cannabinoid is CBLA and the terpene is Beta-Caryophyllene.

In one embodiment, the first cannabinoid is CBLA and the terpene is Alpha-Pinene.

In one embodiment, the first cannabinoid is CBLA and the terpene is Beta-Pinene.

In one embodiment, the first cannabinoid is CBLA and the terpene is Alpha-Bisabolol.

In one embodiment, the first cannabinoid is CBLA and the terpene is Delta-3-Carene.

In one embodiment, the first cannabinoid is CBLA and the terpene is Borneol.

In one embodiment, the first cannabinoid is CBLA and the terpene is Eucalyptol.

In one embodiment, the first cannabinoid is CBLA and the terpene is p-Cymene.

In one embodiment, the first cannabinoid is CBLA and the terpene is Alpha-Terpineol.

In one embodiment, the first cannabinoid is CBLA and the terpene is Alpha-Humulene.

In one embodiment, the first cannabinoid is CBLA and the terpene is Terpinolene.

In one embodiment, the first cannabinoid is CBLA and the terpene is Pulegone.

In one embodiment, the first cannabinoid is CBLA and the terpene is Camphene.

In one embodiment, the first cannabinoid is CBLA and the terpene is Geraniol.

In one embodiment, the first cannabinoid is CBLV and the terpene is Limonene.

In one embodiment, the first cannabinoid is CBLV and the terpene is Nerolidol.

In one embodiment, the first cannabinoid is CBLV and the terpene is Beta-Myrcene.

In one embodiment, the first cannabinoid is CBLV and the terpene is Linalool.

In one embodiment, the first cannabinoid is CBLV and the terpene is Alpha-Caryophyllene.

In one embodiment, the first cannabinoid is CBLV and the terpene is Beta-Caryophyllene.

In one embodiment, the first cannabinoid is CBLV and the terpene is Alpha-Pinene.

In one embodiment, the first cannabinoid is CBLV and the terpene is Beta-Pinene.

In one embodiment, the first cannabinoid is CBLV and the terpene is Alpha-Bisabolol.

In one embodiment, the first cannabinoid is CBLV and the terpene is Delta-3-Carene.

In one embodiment, the first cannabinoid is CBLV and the terpene is Borneol.

In one embodiment, the first cannabinoid is CBLV and the terpene is Eucalyptol.

In one embodiment, the first cannabinoid is CBLV and the terpene is p-Cymene.

In one embodiment, the first cannabinoid is CBLV and the terpene is Alpha-Terpineol.

In one embodiment, the first cannabinoid is CBLV and the terpene is Alpha-Humulene.

In one embodiment, the first cannabinoid is CBLV and the terpene is Terpinolene.

In one embodiment, the first cannabinoid is CBLV and the terpene is Pulegone.

In one embodiment, the first cannabinoid is CBLV and the terpene is Camphene.

In one embodiment, the first cannabinoid is CBLV and the terpene is Geraniol.

In one embodiment, the first cannabinoid is CBLVA and the terpene is Limonene.

In one embodiment, the first cannabinoid is CBLVA and the terpene is Nerolidol.

In one embodiment, the first cannabinoid is CBLVA and the terpene is Beta-Myrcene.

In one embodiment, the first cannabinoid is CBLVA and the terpene is Linalool.

In one embodiment, the first cannabinoid is CBLVA and the terpene is Alpha-Caryophyllene.

In one embodiment, the first cannabinoid is CBLVA and the terpene is Beta-Caryophyllene.

In one embodiment, the first cannabinoid is CBLVA and the terpene is Alpha-Pinene.

In one embodiment, the first cannabinoid is CBLVA and the terpene is Beta-Pinene.

In one embodiment, the first cannabinoid is CBLVA and the terpene is Alpha-Bisabolol.

In one embodiment, the first cannabinoid is CBLVA and the terpene is Delta-3-Carene.

In one embodiment, the first cannabinoid is CBLVA and the terpene is Borneol.

In one embodiment, the first cannabinoid is CBLVA and the terpene is Eucalyptol.

In one embodiment, the first cannabinoid is CBLVA and the terpene is p-Cymene.

In one embodiment, the first cannabinoid is CBLVA and the terpene is Alpha-Terpineol.

In one embodiment, the first cannabinoid is CBLVA and the terpene is Alpha-Humulene.

In one embodiment, the first cannabinoid is CBLVA and the terpene is

Terpinolene.

In one embodiment, the first cannabinoid is CBLVA and the terpene is Pulegone.

In one embodiment, the first cannabinoid is CBLVA and the terpene is Camphene.

In one embodiment, the first cannabinoid is CBLVA and the terpene is Geraniol.

In one embodiment, the first cannabinoid is CBE and the terpene is Limonene.

In one embodiment, the first cannabinoid is CBE and the terpene is Nerolidol.

In one embodiment, the first cannabinoid is CBE and the terpene is Beta-Myrcene.

In one embodiment, the first cannabinoid is CBE and the terpene is Linalool.

In one embodiment, the first cannabinoid is CBE and the terpene is Alpha-Caryophyllene.

In one embodiment, the first cannabinoid is CBE and the terpene is Beta-Caryophyllene.

In one embodiment, the first cannabinoid is CBE and the terpene is Alpha-Pinene.

In one embodiment, the first cannabinoid is CBE and the terpene is Beta-Pinene.

In one embodiment, the first cannabinoid is CBE and the terpene is Alpha-Bisabolol.

In one embodiment, the first cannabinoid is CBE and the terpene is Delta-3-Carene.

In one embodiment, the first cannabinoid is CBE and the terpene is Borneol.

In one embodiment, the first cannabinoid is CBE and the terpene is Eucalyptol.

In one embodiment, the first cannabinoid is CBE and the terpene is p-Cymene.

In one embodiment, the first cannabinoid is CBE and the terpene is Alpha-Terpineol.

In one embodiment, the first cannabinoid is CBE and the terpene is Alpha-Humulene.

In one embodiment, the first cannabinoid is CBE and the terpene is Terpinolene.

In one embodiment, the first cannabinoid is CBE and the terpene is Pulegone.

In one embodiment, the first cannabinoid is CBE and the terpene is Camphene.

In one embodiment, the first cannabinoid is CBE and the terpene is Geraniol.

In one embodiment, the first cannabinoid is CBEA and the terpene is Limonene.

In one embodiment, the first cannabinoid is CBEA and the terpene is Nerolidol.

In one embodiment, the first cannabinoid is CBEA and the terpene is Beta-Myrcene.

In one embodiment, the first cannabinoid is CBEA and the terpene is Linalool.

In one embodiment, the first cannabinoid is CBEA and the terpene is Alpha-Caryophyllene.

In one embodiment, the first cannabinoid is CBEA and the terpene is Beta-Caryophyllene.

In one embodiment, the first cannabinoid is CBEA and the terpene is Alpha-Pinene.

In one embodiment, the first cannabinoid is CBEA and the terpene is Beta-Pinene.

In one embodiment, the first cannabinoid is CBEA and the terpene is Alpha-Bisabolol.

In one embodiment, the first cannabinoid is CBEA and the terpene is Delta-3-Carene.

In one embodiment, the first cannabinoid is CBEA and the terpene is Borneol.

In one embodiment, the first cannabinoid is CBEA and the terpene is Eucalyptol.

In one embodiment, the first cannabinoid is CBEA and the terpene is p-Cymene.

In one embodiment, the first cannabinoid is CBEA and the terpene is Alpha-Terpineol.

In one embodiment, the first cannabinoid is CBEA and the terpene is Alpha-Humulene.

In one embodiment, the first cannabinoid is CBEA and the terpene is Terpinolene.

In one embodiment, the first cannabinoid is CBEA and the terpene is Pulegone.

In one embodiment, the first cannabinoid is CBEA and the terpene is Camphene.

In one embodiment, the first cannabinoid is CBEA and the terpene is Geraniol.

In one embodiment, the first cannabinoid is CBEV and the terpene is Limonene.

In one embodiment, the first cannabinoid is CBEV and the terpene is Nerolidol.

In one embodiment, the first cannabinoid is CBEV and the terpene is Beta-Myrcene.

In one embodiment, the first cannabinoid is CBEV and the terpene is Linalool.

In one embodiment, the first cannabinoid is CBEV and the terpene is Alpha-Caryophyllene.

In one embodiment, the first cannabinoid is CBEV and the terpene is Beta-Caryophyllene.

In one embodiment, the first cannabinoid is CBEV and the terpene is Alpha-Pinene.

In one embodiment, the first cannabinoid is CBEV and the terpene is Beta-Pinene.

In one embodiment, the first cannabinoid is CBEV and the terpene is Alpha-Bisabolol.

In one embodiment, the first cannabinoid is CBEV and the terpene is Delta-3-Carene.

In one embodiment, the first cannabinoid is CBEV and the terpene is Borneol.

In one embodiment, the first cannabinoid is CBEV and the terpene is Eucalyptol.

In one embodiment, the first cannabinoid is CBEV and the terpene is p-Cymene.

In one embodiment, the first cannabinoid is CBEV and the terpene is Alpha-Terpineol.

In one embodiment, the first cannabinoid is CBEV and the terpene is Alpha-Humulene.

In one embodiment, the first cannabinoid is CBEV and the terpene is Terpinolene.

In one embodiment, the first cannabinoid is CBEV and the terpene is Pulegone.

In one embodiment, the first cannabinoid is CBEV and the terpene is Camphene.

In one embodiment, the first cannabinoid is CBEV and the terpene is Geraniol.

In one embodiment, the first cannabinoid is CBEVA and the terpene is Limonene.

In one embodiment, the first cannabinoid is CBEVA and the terpene is Nerolidol.

In one embodiment, the first cannabinoid is CBEVA and the terpene is Beta-Myrcene.

In one embodiment, the first cannabinoid is CBEVA and the terpene is Linalool.

In one embodiment, the first cannabinoid is CBEVA and the terpene is Alpha-Caryophyllene.

In one embodiment, the first cannabinoid is CBEVA and the terpene is Beta-Caryophyllene.

In one embodiment, the first cannabinoid is CBEVA and the terpene is Alpha-Pinene.

In one embodiment, the first cannabinoid is CBEVA and the terpene is Beta-Pinene.

In one embodiment, the first cannabinoid is CBEVA and the terpene is Alpha-Bisabolol.

In one embodiment, the first cannabinoid is CBEVA and the terpene is Delta-3-Carene.

In one embodiment, the first cannabinoid is CBEVA and the terpene is Borneol.

In one embodiment, the first cannabinoid is CBEVA and the terpene is Eucalyptol.

In one embodiment, the first cannabinoid is CBEVA and the terpene is p-Cymene.

In one embodiment, the first cannabinoid is CBEVA and the terpene is Alpha-Terpineol.

In one embodiment, the first cannabinoid is CBEVA and the terpene is Alpha-Humulene.

In one embodiment, the first cannabinoid is CBEVA and the terpene is Terpinolene.

In one embodiment, the first cannabinoid is CBEVA and the terpene is Pulegone.

In one embodiment, the first cannabinoid is CBEVA and the terpene is Camphene.

In one embodiment, the first cannabinoid is CBEVA and the terpene is Geraniol.

In any of the above embodiments comprising specific combinations of a first cannabinoid and a terpene, one or both of the first cannabinoid and the terpene may be purified. In an embodiment, both the first cannabinoid and the terpene are purified.

In any of the above embodiments comprising specific combinations of a first cannabinoid and a terpene, the terpene may be replaced with a second cannabinoid that is different than the first cannabinoid, such that the device comprises a first cannabinoid and a second cannbinoid. In this regard, in an embodiment, the device disclosed herein comprises and is configured for the delivery of unique combinations of a first purified cannabinoid and a second cannabinoid. In an embodiment, the First Composition comprises a first cannabinoid and the Second Composition comprises a second cannabinoid. In an embodiment, one or both of the First Composition and the Second Composition comprise both a first cannabinoid and a second cannabinoid. In one embodiment, the combination of a first cannabinoid and a second cannabinoid provide a synergistic effect at a mammalian cellular receptor, such as a G protein coupled receptor. In one embodiment, the receptor is CB1. In one embodiment, the receptor is CB2. In one embodiment, the receptor is GPR55. In one embodiment, the receptor is 5HT-1A. In one embodiment, the receptor is 5HT-2A. In one embodiment, the receptor is TRPV1. In one embodiment, the receptor is an opioid receptor, e.g., p-opioid receptor.

In an embodiment, the second cannabinoid is chosen from THC, Δ9-THC, Δ8-THC, THCA, THCV, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

Accordingly, in an embodiment, the device as disclosed herein comprises (i) a first cannabinoid chosen from THC, Δ9-THC, Δ8-THC, THCA, THCV, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA; and (ii) a second cannabinoid chosen from a different one of THC, Δ9-THC, Δ8-THC, THCA, THCV, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA than the first cannabinoid, wherein the first and second cannabinoids are in different compositions (First and Second Compositions) or are in the same composition. In an embodiment, the first and second cannabinoids are purified.

In one embodiment, the first cannabinoid is Δ9-THC and the second cannabinoid is Δ8-THC.

In one embodiment, the first cannabinoid is Δ9-THC and the second cannabinoid is THCA.

In one embodiment, the first cannabinoid is Δ9-THC and the second cannabinoid is THCV.

In one embodiment, the first cannabinoid is Δ9-THC and the second cannabinoid is THCVA.

In one embodiment, the first cannabinoid is Δ9-THC and the second cannabinoid is CBD.

In one embodiment, the first cannabinoid is Δ9-THC and the second cannabinoid is CBDA.

In one embodiment, the first cannabinoid is Δ9-THC and the second cannabinoid is CBDV.

In one embodiment, the first cannabinoid is Δ9-THC and the second cannabinoid is CBDVA.

In one embodiment, the first cannabinoid is Δ9-THC and the second cannabinoid is CBC.

In one embodiment, the first cannabinoid is Δ9-THC and the second cannabinoid is CBCA.

In one embodiment, the first cannabinoid is Δ9-THC and the second cannabinoid is CBCV.

In one embodiment, the first cannabinoid is Δ9-THC and the second cannabinoid is CBCVA.

In one embodiment, the first cannabinoid is Δ9-THC and the second cannabinoid is CBG.

In one embodiment, the first cannabinoid is Δ9-THC and the second cannabinoid is CBGA.

In one embodiment, the first cannabinoid is Δ9-THC and the second cannabinoid is CBGV.

In one embodiment, the first cannabinoid is Δ9-THC and the second cannabinoid is CBGVA.

In one embodiment, the first cannabinoid is Δ9-THC and the second cannabinoid is CBN.

In one embodiment, the first cannabinoid is Δ9-THC and the second cannabinoid is CBNA.

In one embodiment, the first cannabinoid is Δ9-THC and the second cannabinoid is CBNV.

In one embodiment, the first cannabinoid is Δ9-THC and the second cannabinoid is CBNVA.

In one embodiment, the first cannabinoid is Δ8-THC and the second cannabinoid is THCA.

In one embodiment, the first cannabinoid is Δ8-THC and the second cannabinoid is THCV.

In one embodiment, the first cannabinoid is Δ8-THC and the second cannabinoid is THCVA.

In one embodiment, the first cannabinoid is Δ8-THC and the second cannabinoid is CBD.

In one embodiment, the first cannabinoid is Δ8-THC and the second cannabinoid is CBDA.

In one embodiment, the first cannabinoid is Δ8-THC and the second cannabinoid is CBDV.

In one embodiment, the first cannabinoid is Δ8-THC and the second cannabinoid is CBDVA.

In one embodiment, the first cannabinoid is Δ8-THC and the second cannabinoid is CBC.

In one embodiment, the first cannabinoid is Δ8-THC and the second cannabinoid is CBCA.

In one embodiment, the first cannabinoid is Δ8-THC and the second cannabinoid is CBCV.

In one embodiment, the first cannabinoid is Δ8-THC and the second cannabinoid is CBCVA.

In one embodiment, the first cannabinoid is Δ8-THC and the second cannabinoid is CBG.

In one embodiment, the first cannabinoid is Δ8-THC and the second cannabinoid is CBGA.

In one embodiment, the first cannabinoid is Δ8-THC and the second cannabinoid is CBGV.

In one embodiment, the first cannabinoid is Δ8-THC and the second cannabinoid is CBGVA.

In one embodiment, the first cannabinoid is Δ8-THC and the second cannabinoid is CBN.

In one embodiment, the first cannabinoid is Δ8-THC and the second cannabinoid is CBNA.

In one embodiment, the first cannabinoid is Δ8-THC and the second cannabinoid is CBNV.

In one embodiment, the first cannabinoid is Δ8-THC and the second cannabinoid is CBNVA.

In one embodiment, the first cannabinoid is THCA and the second cannabinoid is THCV.

In one embodiment, the first cannabinoid is THCA and the second cannabinoid is THCVA.

In one embodiment, the first cannabinoid is THCA and the second cannabinoid is CBD.
In one embodiment, the first cannabinoid is THCA and the second cannabinoid is CBDA.
In one embodiment, the first cannabinoid is THCA and the second cannabinoid is CBDV.

In one embodiment, the first cannabinoid is THCA and the second cannabinoid is CBDVA.

In one embodiment, the first cannabinoid is THCA and the second cannabinoid is CBC.
In one embodiment, the first cannabinoid is THCA and the second cannabinoid is CBCA.
In one embodiment, the first cannabinoid is THCA and the second cannabinoid is CBCV.

In one embodiment, the first cannabinoid is THCA and the second cannabinoid is CBCVA.

In one embodiment, the first cannabinoid is THCA and the second cannabinoid is CBG.
In one embodiment, the first cannabinoid is THCA and the second cannabinoid is CBGA.
In one embodiment, the first cannabinoid is THCA and the second cannabinoid is CBGV.

In one embodiment, the first cannabinoid is THCA and the second cannabinoid is CBGVA.

In one embodiment, the first cannabinoid is THCA and the second cannabinoid is CBN.
In one embodiment, the first cannabinoid is THCA and the second cannabinoid is CBNA.
In one embodiment, the first cannabinoid is THCA and the second cannabinoid is CBNV.

In one embodiment, the first cannabinoid is THCA and the second cannabinoid is CBNVA.

In one embodiment, the first cannabinoid is THCV and the second cannabinoid is THCVA.

In one embodiment, the first cannabinoid is THCV and the second cannabinoid is CBD.
In one embodiment, the first cannabinoid is THCV and the second cannabinoid is CBDA.
In one embodiment, the first cannabinoid is THCV and the second cannabinoid is CBDV.

In one embodiment, the first cannabinoid is THCV and the second cannabinoid is CBDVA.

In one embodiment, the first cannabinoid is THCV and the second cannabinoid is CBC.
In one embodiment, the first cannabinoid is THCV and the second cannabinoid is CBCA.
In one embodiment, the first cannabinoid is THCV and the second cannabinoid is CBCV.

In one embodiment, the first cannabinoid is THCV and the second cannabinoid is CBCVA.

In one embodiment, the first cannabinoid is THCV and the second cannabinoid is CBG.
In one embodiment, the first cannabinoid is THCV and the second cannabinoid is CBGA.
In one embodiment, the first cannabinoid is THCV and the second cannabinoid is CBGV.

In one embodiment, the first cannabinoid is THCV and the second cannabinoid is CBGVA.

In one embodiment, the first cannabinoid is THCV and the second cannabinoid is CBN.
In one embodiment, the first cannabinoid is THCV and the second cannabinoid is CBNA.
In one embodiment, the first cannabinoid is THCV and the second cannabinoid is CBNV.

In one embodiment, the first cannabinoid is THCV and the second cannabinoid is CBNVA.

In one embodiment, the first cannabinoid is THCVA and the second cannabinoid is CBD.

In one embodiment, the first cannabinoid is THCVA and the second cannabinoid is CBDA.

In one embodiment, the first cannabinoid is THCVA and the second cannabinoid is CBDV.

In one embodiment, the first cannabinoid is THCVA and the second cannabinoid is CBDVA.

In one embodiment, the first cannabinoid is THCVA and the second cannabinoid is CBC.

In one embodiment, the first cannabinoid is THCVA and the second cannabinoid is CBCA.

In one embodiment, the first cannabinoid is THCVA and the second cannabinoid is CBCV.

In one embodiment, the first cannabinoid is THCVA and the second cannabinoid is CBCVA.

In one embodiment, the first cannabinoid is THCVA and the second cannabinoid is CBG.

In one embodiment, the first cannabinoid is THCVA and the second cannabinoid is CBGA.

In one embodiment, the first cannabinoid is THCVA and the second cannabinoid is CBGV.

In one embodiment, the first cannabinoid is THCVA and the second cannabinoid is CBGVA.

In one embodiment, the first cannabinoid is THCVA and the second cannabinoid is CBN.

In one embodiment, the first cannabinoid is THCVA and the second cannabinoid is CBNA.

In one embodiment, the first cannabinoid is THCVA and the second cannabinoid is CBNV.

In one embodiment, the first cannabinoid is THCVA and the second cannabinoid is CBNVA.

In one embodiment, the first cannabinoid is CBD and the second cannabinoid is CBDA.
In one embodiment, the first cannabinoid is CBD and the second cannabinoid is CBDV.
In one embodiment, the first cannabinoid is CBD and the second cannabinoid is CBDVA.
In one embodiment, the first cannabinoid is CBD and the second cannabinoid is CBC.
In one embodiment, the first cannabinoid is CBD and the second cannabinoid is CBCA.
In one embodiment, the first cannabinoid is CBD and the second cannabinoid is CBCV.
In one embodiment, the first cannabinoid is CBD and the second cannabinoid is CBCVA.
In one embodiment, the first cannabinoid is CBD and the second cannabinoid is CBG.
In one embodiment, the first cannabinoid is CBD and the second cannabinoid is CBGA.
In one embodiment, the first cannabinoid is CBD and the second cannabinoid is CBGV.
In one embodiment, the first cannabinoid is CBD and the second cannabinoid is CBGVA.
In one embodiment, the first cannabinoid is CBD and the second cannabinoid is CBN.
In one embodiment, the first cannabinoid is CBD and the second cannabinoid is CBNA.
In one embodiment, the first cannabinoid is CBD and the second cannabinoid is CBNV.
In one embodiment, the first cannabinoid is CBD and the second cannabinoid is CBNVA.
In one embodiment, the first cannabinoid is CBDA and the second cannabinoid is CBDV.
In one embodiment, the first cannabinoid is CBDA and the second cannabinoid is CBDVA.
In one embodiment, the first cannabinoid is CBDA and the second cannabinoid is CBC.
In one embodiment, the first cannabinoid is CBDA and the second cannabinoid is CBCA.
In one embodiment, the first cannabinoid is CBDA and the second cannabinoid is CBCV.

In one embodiment, the first cannabinoid is CBDA and the second cannabinoid is CBCVA.

In one embodiment, the first cannabinoid is CBDA and the second cannabinoid is CBG.
In one embodiment, the first cannabinoid is CBDA and the second cannabinoid is CBGA.
In one embodiment, the first cannabinoid is CBDA and the second cannabinoid is CBGV.

In one embodiment, the first cannabinoid is CBDA and the second cannabinoid is CBGVA.

In one embodiment, the first cannabinoid is CBDA and the second cannabinoid is CBN.
In one embodiment, the first cannabinoid is CBDA and the second cannabinoid is CBNA.
In one embodiment, the first cannabinoid is CBDA and the second cannabinoid is CBNV.

In one embodiment, the first cannabinoid is CBDA and the second cannabinoid is CBNVA.

In one embodiment, the first cannabinoid is CBDV and the second cannabinoid is CBDVA.

In one embodiment, the first cannabinoid is CBDV and the second cannabinoid is CBC.
In one embodiment, the first cannabinoid is CBDV and the second cannabinoid is CBCA.
In one embodiment, the first cannabinoid is CBDV and the second cannabinoid is CBCV.

In one embodiment, the first cannabinoid is CBDV and the second cannabinoid is CBCVA.

In one embodiment, the first cannabinoid is CBDV and the second cannabinoid is CBG.
In one embodiment, the first cannabinoid is CBDV and the second cannabinoid is CBGA.
In one embodiment, the first cannabinoid is CBDV and the second cannabinoid is CBGV.

In one embodiment, the first cannabinoid is CBDV and the second cannabinoid is CBGVA.

In one embodiment, the first cannabinoid is CBDV and the second cannabinoid is CBN.
In one embodiment, the first cannabinoid is CBDV and the second cannabinoid is CBNA.
In one embodiment, the first cannabinoid is CBDV and the second cannabinoid is CBNV.

In one embodiment, the first cannabinoid is CBDV and the second cannabinoid is CBNVA.

In one embodiment, the first cannabinoid is CBDVA and the second cannabinoid is CBC.

In one embodiment, the first cannabinoid is CBDVA and the second cannabinoid is CBCA.

In one embodiment, the first cannabinoid is CBDVA and the second cannabinoid is CBCV.

In one embodiment, the first cannabinoid is CBDVA and the second cannabinoid is CBCVA.

In one embodiment, the first cannabinoid is CBDVA and the second cannabinoid is CBG.

In one embodiment, the first cannabinoid is CBDVA and the second cannabinoid is CBGA.

In one embodiment, the first cannabinoid is CBDVA and the second cannabinoid is CBGV.

In one embodiment, the first cannabinoid is CBDVA and the second cannabinoid is CBGVA.
In one embodiment, the first cannabinoid is CBDVA and the second cannabinoid is CBN.

In one embodiment, the first cannabinoid is CBDVA and the second cannabinoid is CBNA.

In one embodiment, the first cannabinoid is CBDVA and the second cannabinoid is CBNV.

In one embodiment, the first cannabinoid is CBDVA and the second cannabinoid is CBNVA.

In one embodiment, the first cannabinoid is CBC and the second cannabinoid is CBCA.
In one embodiment, the first cannabinoid is CBC and the second cannabinoid is CBCV.
In one embodiment, the first cannabinoid is CBC and the second cannabinoid is CBCVA.
In one embodiment, the first cannabinoid is CBC and the second cannabinoid is CBG.
In one embodiment, the first cannabinoid is CBC and the second cannabinoid is CBGA.
In one embodiment, the first cannabinoid is CBC and the second cannabinoid is CBGV.
In one embodiment, the first cannabinoid is CBC and the second cannabinoid is CBGVA.
In one embodiment, the first cannabinoid is CBC and the second cannabinoid is CBN.
In one embodiment, the first cannabinoid is CBC and the second cannabinoid is CBNA.
In one embodiment, the first cannabinoid is CBC and the second cannabinoid is CBNV.
In one embodiment, the first cannabinoid is CBC and the second cannabinoid is CBNVA.
In one embodiment, the first cannabinoid is CBCA and the second cannabinoid is CBCV.

In one embodiment, the first cannabinoid is CBCA and the second cannabinoid is CBCVA.

In one embodiment, the first cannabinoid is CBCA and the second cannabinoid is CBG.
In one embodiment, the first cannabinoid is CBCA and the second cannabinoid is CBGA.
In one embodiment, the first cannabinoid is CBCA and the second cannabinoid is CBGV.

In one embodiment, the first cannabinoid is CBCA and the second cannabinoid is CBGVA.

In one embodiment, the first cannabinoid is CBCA and the second cannabinoid is CBN.
In one embodiment, the first cannabinoid is CBCA and the second cannabinoid is CBNA.
In one embodiment, the first cannabinoid is CBCA and the second cannabinoid is CBNV.

In one embodiment, the first cannabinoid is CBCA and the second cannabinoid is CBNVA.

In one embodiment, the first cannabinoid is CBCV and the second cannabinoid is CBCVA.
In one embodiment, the first cannabinoid is CBCV and the second cannabinoid is CBG.
In one embodiment, the first cannabinoid is CBCV and the second cannabinoid is CBGA.
In one embodiment, the first cannabinoid is CBCV and the second cannabinoid is CBGV.

In one embodiment, the first cannabinoid is CBCV and the second cannabinoid is CBGVA.

In one embodiment, the first cannabinoid is CBCV and the second cannabinoid is CBN.
In one embodiment, the first cannabinoid is CBCV and the second cannabinoid is CBNA.
In one embodiment, the first cannabinoid is CBCV and the second cannabinoid is CBNV.

In one embodiment, the first cannabinoid is CBCV and the second cannabinoid is CBNVA.

In one embodiment, the first cannabinoid is CBCV and the second cannabinoid is CBCA.
In one embodiment, the first cannabinoid is CBCVA and the second cannabinoid is CBG.
In one embodiment, the first cannabinoid is CBCVA and the second cannabinoid is CBGA.

In one embodiment, the first cannabinoid is CBCVA and the second cannabinoid is CBGV.

In one embodiment, the first cannabinoid is CBCVA and the second cannabinoid is CBGVA.

In one embodiment, the first cannabinoid is CBCVA and the second cannabinoid is CBN.

In one embodiment, the first cannabinoid is CBCVA and the second cannabinoid is CBNA.

In one embodiment, the first cannabinoid is CBCVA and the second cannabinoid is CBNV.

In one embodiment, the first cannabinoid is CBCVA and the second cannabinoid is CBNVA.

In one embodiment, the first cannabinoid is CBG and the second cannabinoid is CBGA.
In one embodiment, the first cannabinoid is CBG and the second cannabinoid is CBGV.
In one embodiment, the first cannabinoid is CBG and the second cannabinoid is CBGVA.
In one embodiment, the first cannabinoid is CBG and the second cannabinoid is CBN.
In one embodiment, the first cannabinoid is CBG and the second cannabinoid is CBNA.
In one embodiment, the first cannabinoid is CBG and the second cannabinoid is CBNV.
In one embodiment, the first cannabinoid is CBG and the second cannabinoid is CBNVA.
In one embodiment, the first cannabinoid is CBGA and the second cannabinoid is CBGV.

In one embodiment, the first cannabinoid is CBGA and the second cannabinoid is CBGVA.

In one embodiment, the first cannabinoid is CBGA and the second cannabinoid is CBN.
In one embodiment, the first cannabinoid is CBGA and the second cannabinoid is CBNA.
In one embodiment, the first cannabinoid is CBGA and the second cannabinoid is CBNV.

In one embodiment, the first cannabinoid is CBGA and the second cannabinoid is CBNVA.

In one embodiment, the first cannabinoid is CBGV and the second cannabinoid is CBN.
In one embodiment, the first cannabinoid is CBGV and the second cannabinoid is CBNA.
In one embodiment, the first cannabinoid is CBGV and the second cannabinoid is CBNV.

In one embodiment, the first cannabinoid is CBGV and the second cannabinoid is CBNVA.

In one embodiment, the first cannabinoid is CBGVA and the second cannabinoid is CBN.

In one embodiment, the first cannabinoid is CBGVA and the second cannabinoid is CBNA.

In one embodiment, the first cannabinoid is CBGVA and the second cannabinoid is CBNV.

In one embodiment, the first cannabinoid is CBGVA and the second cannabinoid is CBNVA.

In one embodiment, the first cannabinoid is CBN and the second cannabinoid is CBNA.
In one embodiment, the first cannabinoid is CBN and the second cannabinoid is CBNV.
In one embodiment, the first cannabinoid is CBN and the second cannabinoid is CBNVA.
In one embodiment, the first cannabinoid is CBNA and the second cannabinoid is CBNV.

In one embodiment, the first cannabinoid is CBNA and the second cannabinoid is CBNVA.

In one embodiment, the first cannabinoid is CBNV and the second cannabinoid is CBNVA.

In any of the above embodiments comprising specific combinations of a first cannabinoid and a second cannabinoid, one or both of the first cannabinoid and the second cannabinoid may be purified. In an embodiment, both the first cannabinoid and the second cannabinoid are purified.

In any of the above embodiments comprising specific combinations of a first cannabinoid and a second cannabinoid, a terpene may further be included. In an embodiment, the terpene is chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol. In an embodiment, the terpene is in a different composition than the cannabinoids. In an embodiment, the terpene is in the same composition as at least one of the cannabinoids. In an embodiment, the terpene is purified.

In any of the above embodiments comprising specific combinations of a first cannabinoid and a second cannabinoid, a third cannabinoid may be included. In an embodiment, the third cannabinoid is chosen from THC, Δ9-THC, Δ8-THC, THCA, THCV, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA. In an embodiment, the third cannabinoid is in a different composition than the other cannabinoids. In an embodiment, the third cannabinoid is in the same composition as at least one of the cannabinoids. In an embodiment, the third cannabinoid is purified.

In another aspect, disclosed herein are new methods for administering one or more Active Ingredients. The methods comprise employing the device as disclosed herein.

In one embodiment, the methods disclosed herein comprise administering one or more Active Ingredients. In one embodiment, the methods disclosed herein comprise administering two or more Active Ingredients at the same time. In one embodiment, the methods disclosed herein comprise administering two or more compositions at the same time. In one embodiment, the methods disclosed herein do not comprise heating the Active Ingredients within the devices disclosed herein, prior to administration.

In some embodiments, the methods disclosed herein are for administering cannabinoids. In one embodiment, the methods disclosed herein comprise administering one or more cannabinoids in precise doses. In one embodiment, the methods disclosed herein comprise administering one or more cannabinoids with other Active Ingredients, e.g., terpenes. In one embodiment, the methods disclosed herein comprise administering one or more cannabinoids without producing smoke by using a device as disclosed herein. In one embodiment, the methods disclosed herein comprise administering one or more cannabinoids without heating the cannabinoids within the device disclosed herein.

In one embodiment, disclosed herein is a method of administering an aqueous composition, comprising providing a device as disclosed herein, and administering one or Active Ingredients to a subject using the device. In an embodiment, the Active Ingredients are cannabinoids.

In a particular embodiment, disclosed herein is a method of administering an aqueous composition, comprising:
providing a device comprising: (i) a Container comprising a First Chamber and a Second Chamber; wherein the First Chamber contains a First Composition comprising an Aqueous Acid; wherein the Second Chamber contains a Second Composition comprising an Aqueous Carbonate Base; wherein at least one of the First Composition and the Second Composition comprises an Active Ingredient; (ii) a means for expelling the First Composition from the First Chamber; (iii) a means for expelling the Second Composition from the Second Chamber; and (iv) a Nozzle,
expelling the First Composition from the First Chamber;
expelling the Second Composition from the Second Chamber; and
administering the First Composition and the Second Composition through the Nozzle.

As used herein, the term “expelling the Composition from the Chamber” refers to forcing, pushing, and/or distributing a solution or formulation from a repository, e.g., a Container, a Chamber, a bag, a reservoir, etc. In one embodiment, expelling a Composition from a Chamber comprises forcing a solution through a Nozzle with a pump system. In one embodiment, expelling a Composition from a Chamber comprises connecting a Tube to a Nozzle and forcing a solution from the Tube through the Nozzle. In one embodiment, expelling a Composition from a Chamber comprises aerosolizing a Composition into a mist with an atomizer. In one embodiment, the methods disclosed herein comprise expelling a First Composition from a First Chamber. In one embodiment, the methods disclosed herein comprise expelling a Second Composition from a Second Chamber.

As used herein, term “administering” refers to dispensing, applying, and/or treating an subject with a compound or compounds. In an embodiment, the subject is a human. In one embodiment, administering comprises dispensing a composition from a Container, Chamber, bag, etc. In one embodiment, administering comprises expelling an Active Ingredient. In one embodiment, administering comprises expelling two or more Active Ingredients at the same time. In one embodiment, administering comprises expelling two or more compounds at the same time to create an effervescent.

In one embodiment, the methods disclosed herein comprise administering a First Composition through a Nozzle. In one embodiment, the methods disclosed herein comprise administering a Second Composition through a Nozzle. In one embodiment, the methods disclosed herein comprise administering a First Composition and a Second Composition through a Nozzle.

In an embodiment, the methods disclosed herein further comprise formulating the First Composition comprising the Aqueous Acid and formulating the Second Composition comprising the Aqueous Carbonate Base. For example, in an embodiment, disclosed herein is a method of administering an aqueous composition, comprising:
formulating a First Composition having an Aqueous Acid and formulating a Second Composition having an Aqueous Carbonate Base; wherein at least one of the First Composition and the Second Composition comprises an Active Ingredient and wherein a Container comprises a First Chamber and a Second Chamber; wherein the First Chamber comprises the First Composition and the Second Chamber comprises the Second composition;
expelling the First Composition from the First Chamber;
expelling the Second Composition from the Second Chamber; and
administering the First Composition and the Second Composition through a Nozzle.

As used herein, the “formulating a Composition” refers to creating, mixing, making, and/or devising a chemical formulation. In one embodiment, formulating a Composition comprises mixing one or more chemical compounds. In one embodiment, formulating a Composition comprises mixing an Active Ingredient with an aqueous composition, e.g., an Aqueous Acid, an Aqueous Carbonate Base, etc. In one embodiment, formulating a Composition comprises stabilizing an Active Ingredient, e.g., mixing a pH buffer with an aqueous solution, mixing an antioxidant with an Active Ingredient, etc. In one embodiment, the methods disclosed herein comprise formulating a First Composition. In one embodiment, the methods disclosed herein comprise formulating a Second Composition. In one embodiment, the methods disclosed herein comprise formulating two or more Compositions. In one embodiment, the methods disclosed herein comprise formulating a First Composition with an Active Ingredient. In one embodiment, the methods disclosed herein comprise formulating a Second Composition with an Active Ingredient. In one embodiment, the methods disclosed herein comprise formulating a First Composition with an Active Ingredient and a Second Composition with an Active Ingredient. In one embodiment, the methods disclosed herein comprise formulating a First Composition with an Aqueous Acid. In one embodiment, the methods disclosed herein comprise formulating a Second Composition with an Aqueous Carbonate Base.

In one embodiment, the methods disclosed herein comprise administering about a 1:1-20:1 molar ratio of a First Active Ingredient to a Second Active Ingredient.

In one embodiment, the methods disclosed herein comprise administering about a 1:1-15:1 molar ratio of a First Active Ingredient to a Second Active Ingredient.

In one embodiment, the methods disclosed herein comprise administering about a 1:1-10:1 molar ratio of a First Active Ingredient to a Second Active Ingredient.

In one embodiment, the methods disclosed herein comprise administering about a 1:1-5:1 molar ratio of a First Active Ingredient to a Second Active Ingredient.

In one embodiment, the methods disclosed herein comprise administering about a 1:1 molar ratio of a First Active Ingredient to a Second Active Ingredient.

In one embodiment, the methods disclosed herein comprise administering about a 1:1 to 1:25 molar ratio of the concentration of a First Composition to the concentration of a Second Composition.

In one embodiment, the methods disclosed herein comprise administering about a 1:1 to 1:20 molar ratio of the concentration of a First Composition to the concentration of a Second Composition.

In one embodiment, the methods disclosed herein comprise administering about a 1:1 to 1:15 molar ratio of the concentration of a First Composition to the concentration of a Second Composition.

In one embodiment, the methods disclosed herein comprise administering about a 1:1 to 1:10 molar ratio of the concentration of a First Composition to the concentration of a Second Composition.

In one embodiment, the methods disclosed herein comprise administering about a 1:1 to 1:5 molar ratio of the concentration of a First Composition to the concentration of a Second Composition.

In one embodiment, the methods disclosed herein comprise administering about a 1:1 molar ratio of the concentration of a First Composition to the concentration of a Second Composition.

In one embodiment of the methods disclosed herein, the Active Ingredient is a cannabinoid and the methods may involve administering one or more cannabinoids. In an embodiment, each of the First Composition and the Second Composition may individually comprise one or more cannabinoids. In an embodiment, only one of the First Composition or the Second Composition comprises cannabinoid(s). In an embodiment, only one of the First Composition or the Second Composition comprises cannabinoid(s), and it is only a single type of cannabinoid (e.g a first cannabinoid) In an embodiment, each of the First Composition and the Second Composition comprise only a single cannabinoid, and that cannabinoid may be the same in both (e.g. a first cannabinoid) or different (e.g., a first cannabinoid in one and a second cannabinoid in the other).

In another aspect, disclosed herein is the use of the device as disclosed herein for administering one or more Active Ingredients. In an embodiment, the Active Ingredient is a cannabinoid or a combination thereof. In an embodiment, the Active Ingredient is a cannabinoid and a terpene. In an embodiment, the Active Ingredient (e.g. cannabinoid, terpene, etc.) is purified.

Although the present invention herein has been described with reference to various exemplary embodiments, it is to be understood that these embodiments are merely illustrative of the principles and applications of the present invention. Those having skill in the art would recognize that various modifications to the exemplary embodiments may be made, without departing from the scope of the invention.

Moreover, it should be understood that various features and/or characteristics of differing embodiments herein may be combined with one another. In various embodiments, salts, acids, esters, ethers, stereoisomers, enantiomers, various alpha, beta, gamma, trans, cis, etc., forms are all disclosed as well. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

1. A device for administering an aqueous composition, the device comprising:
a container comprising a first chamber and a second chamber;
wherein the first chamber contains a first composition comprising an aqueous acid;
wherein the second chamber contains a second composition comprising an aqueous carbonate base;
wherein at least one of the first composition and the second composition comprises an active ingredient, wherein the active ingredient is a cannabinoid;
a means for expelling the first composition from the first chamber;
a means for expelling the second composition from the second chamber; and
a nozzle.

2. (canceled)

3. The device of claim 1, wherein the cannabinoid is purified.

4. The device of claim 3, wherein the purified cannabinoid is THC, Δ9-THC, Δ8-THC, THCA, THCV, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

5. (canceled)

6. (canceled)

7. (canceled)

8. The device of claim 1, wherein the first composition comprises a first active ingredient and the second composition comprises a second active ingredient, wherein at least one of the first active ingredient and the second active ingredient is the cannabinoid.

9. The device of claim 8, wherein the first active ingredient and the second active ingredient are the same.

10. The device of claim 8, wherein the first active ingredient is the cannabinoid and the second active ingredient is a terpene.

11. The device of claim 8, comprising about a 1:1 molar ratio of the first active ingredient to the second active ingredient.

12. The device of claim 8, wherein the concentration of the first active ingredient is within 0-5% of the concentration of the second active ingredient.

13. The device of claim 1, wherein at least one of the first composition or the second composition comprises Vitamin E TPGS.

14. The device of claim 1, comprising about a 1:1 molar ratio of the concentration of the first composition to the concentration of the second composition.

15. The device of claim 1, wherein the aqueous acid is about 0.5-1.5 mass % of the first composition.

16. The device of claim 1, wherein the aqueous carbonate base is about 0.5-1.5 mass % of the second composition.

17. The device of claim 1, wherein the means for expelling the first composition from the first chamber comprises a first tube attached to the nozzle and extending into the first chamber.

18. The device of claim 1, wherein the means for expelling the second composition from the second chamber comprises a second tube attached to the nozzle and extending into the second chamber.

19. A method of administering an aqueous composition, comprising:
providing a device comprising:
a container comprising a first chamber and a second chamber;
wherein the first chamber contains a first composition comprising an aqueous acid;
wherein the second chamber contains a second composition having an aqueous carbonate base;
wherein at least one of the first composition and the second composition comprises an active ingredient, wherein the active ingredient is a cannabinoid;
a means for expelling the first composition from the first chamber;
a means for expelling the second composition from the second chamber; and
a nozzle,
expelling the first composition from the first chamber;
expelling the second composition from the second chamber; and
administering the first composition and the second composition through the nozzle.

20. (canceled)

21. (canceled)

22. The method of claim 19, wherein the cannabinoid is purified.

23. The method of claim 22, wherein the purified cannabinoid is THC, Δ9-THC, Δ8-THC, THCA, THCV, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

24. (canceled)

25. (canceled)

26. (canceled)

27. The method of claim 19, wherein the first composition comprises a first active ingredient and the second composition comprises a second active ingredient, wherein at least one of the first active ingredient and the second active ingredient is the cannabinoid.

28. The method of claim 27, wherein the first active ingredient and the second active ingredient are the same.

29. The method of claim 27, wherein the first active ingredient is the cannabinoid and the second active ingredient is a terpene.

30. (canceled)

31. (canceled)

32. The method of claim 19, wherein at least one of the first composition or the second composition comprises Vitamin E TPGS.

33. (canceled)

34. (canceled)

35. (canceled)

36. (canceled)

37. (canceled)

38. (canceled)

39. (canceled)

40. (canceled)

41. The device of claim 10, wherein the terpene is purified.

42. The device of claim 41, wherein the purified terpene is chosen from Bornyl Acetate, Alpha-Bisabolol, Borneol, Camphene, Camphor, Carene, Beta-Caryophyllene, Cedrene, Cymene, Elemene, Eucalyptol, Eudesmol, Farnesene, Fenchol, Geraniol, Guaiacol, Humulene, Isoborneol, Limonene, Linalool, Menthol, Beta-Myrcene, Nerolidol, Ocimene, Phellandrene, Phytol, Pinene, Pulegone, Sabinene, Terpineol, Terpinolene, or Valencene.
